(19) United States
(12) Patent Application Publication
(10) Pub. No.: US 2013/0157328 A1
(43) Pub. Date: Jun. 20, 2013

(54) CATALYTIC DEHYDRATION OF LACTIC ACID AND LACTIC ACID ESTERS

(71) Applicants: Cenan Ozmeral, Boston, MA (US); Joseph P. Glas, Sheldon, SC (US); Rajesh Dasari, Lexington, MA (US); Setrak Tanielyan, Maplewood, NJ (US); Ramesh Deoram Bhagat, Newark, NJ (US); Mohan Reddy Kasireddy, North Brunswick, NJ (US)

(72) Inventors: Cenan Ozmeral, Boston, MA (US); Joseph P. Glas, Sheldon, SC (US); Rajesh Dasari, Lexington, MA (US); Setrak Tanielyan, Maplewood, NJ (US); Ramesh Deoram Bhagat, Newark, NJ (US); Mohan Reddy Kasireddy, North Brunswick, NJ (US)

(73) Assignee: Myriant Corporation, Quincy, MA (US)

(21) Appl. No.: 13/819,035

(22) PCT Filed: Sep. 7, 2011
(86) PCT No.: PCT/US11/50707

§ 371 Date: Feb. 26, 2013

Related U.S. Application Data
(60) Provisional application No. 61/402,913, filed on Sep. 7, 2010.

(51) Int. Cl. C12P 7/62

(52) U.S. Cl. CPC C12P 7/62
USPC 435/135

(57) ABSTRACT

This invention relates to catalytic dehydration of lactic acid derived from biological fermentation and its esters into acrylic acid and acrylic acid esters respectively. Disclosed in this invention are chemical catalysts suitable for industrial scale production of acrylic acid and acrylic acid esters. This invention also provides an industrial scale integrated process technology for producing acrylic acid and acrylic acid esters from biological fermentation using renewable resources and biological catalysts.

### CROSS-REFERENCE TO RELATED APPLICATION

This application claims the priority of the U.S. Provisional Application Ser. No. 61/402,913, filed on Sep. 7, 2010.

### FIELD OF THE INVENTION

The present invention is in the field of producing acrylic acid and its derivatives from lactic acid and lactic acid derivatives manufactured from the fermentation of biological feedstock.

### BACKGROUND OF THE INVENTION

Lactic acid, 2-hydroxy-propionic acid (also known as α-hydroxy-propionic acid), is one of the commodity chemicals produced from biomass through fermentation at low cost. Lactic acid possesses a hydroxyl group and a carboxyl group. The presence of two different functional groups makes lactic acid an attractive feedstock for the production of number of commodity organic chemicals such as poly L-lactic acid, acrylic acid, 2,3-pentanedione, pyruvic acid, propionic acid, 1,2-propanediol, acetaldehyde, dilactide and alkyl lactate which are traditionally derived from petrochemical feedstock. The increase in the oil price accompanied by an increase in the efficiency of production of lactic acid through biological fermentation using renewable resources has made the conversion of lactic acid to various commodity chemicals more attractive. It has become an economically viable option for commercial production of acrylic acid using lactic acid derived from biological resources.

Acrylic acid, an α,β unsaturated acid is one of the commodity chemicals that can be derived from lactic acid through a single-step catalytic dehydration. During 2010 about 4,400,000 metric tons of acrylic acid and about 3,900,000 metric tons of acrylate ester were produced from petrochemical feedstock by two-step gas-phase oxidation of propylene. Acrylic acid is used in the manufacture of polymeric flocculants, super absorbents, dispersants, coatings, paints, adhesives, paper products, construction chemicals, water treatment chemicals, and binders for leather, paper and textile.

Acrylic acid can also be derived from the dehydration of 3-hydroxy propionic acid. Currently efforts are being made to produce 3-hydroxypropionic acid using biocatalysts (Straathof et al 2005; Lunelli et al 2008; Jiang et al 2009). However, when compared to the current fermentative production of lactic acid in the commercial scale, the fermentative production of 3-hydroxy propionic acid is still in its developmental stage. When the technology for of 3-hydroxy propionic acid from biological feedstock becomes a commercial reality, the inventions described and claimed in the present patent application can be used effectively to produce acrylic acid and acrylic acid derivatives from 3-hydroxy propionic acid,

The objective of the present invention is to provide an efficient catalytic process for the production of acrylic acid and acrylic esters from lactic acid and its derivatives obtained from the fermentation processes using inexpensive renewable biological feedstock. More specifically, the present invention is focused on identifying cost-effective and scalable manufacturing processes suitable for the dehydration of lactic acid and various esters of lactic acid into acrylic acid and acrylic acid esters respectively in a commercial scale.

The production of acrylic acid and acrylic acid esters from lactic acid and lactic acid esters respectively involves the removal of hydroxyl group from alpha carbon atom and a hydrogen atom from the adjacent beta carbon atom. Thus it would appear that the efficiency of this chemical conversion would depend on the rate constant for the dehydration process. But in reality, the overall efficiency for the dehydration of lactic acid and lactic acid esters depends on inhibiting the competing reactions. For example, under the conditions suitable for the dehydration reaction, the lactic acid molecule tends to form lactide, a dimer of lactic acid resulting from the self-esterification reaction. The lactide molecule readily decomposes into carbon monoxide, acetaldehyde, and water.

Efforts have been made to produce acrylic acid through dehydration of lactic acid at supercritical or near-critical water. Initial experiments were conducted at 385° C. and 34.5 MPa, with an initial lactic acid concentration of 0.1 mol·L⁻¹ and residence time of approximately 30 seconds. The results from this experiment indicated that decarbonylation to acetaldehyde was higher with the addition of H₂SO₄ and acrylic acid production was enhanced with the addition of NaOH (Mok and Antal Jr., 1989). Lira and McCracken (1993) have reported that the addition of small amount of (<0.01 mol·L−1) of Na₂HPO₄ to the 0.4 mol·L⁻¹ reactant solution raised the pH value and increased the acrylic acid molar yield from 35% to higher than 58% on the basis of conversion of lactic acid. Apparently, the addition of Na₂HPO₄ provided moderate enhancement of the rate constant for acrylic acid production while suppressing the rate constant for the competing decarbonylation, decarboxylation, and secondary reactions. Recently Aida et al (2009) investigated the reaction of lactic acid with a flow apparatus in water at high temperature (450° C.) and high pressures (40-100 MPa). Acrylic acid and acetaldehyde were produced as major products in this reaction. Acetic acid and propionic acid were obtained as the minor products. The maximum selectivity of acrylic acid was 44% at 23% lactic acid conversion with a residence time of 0.8 seconds. The data and the kinetic analysis consistently showed that both dehydration and the combined decarboxylation and decarbonylation reactions continue to be promoted in supercritical water as pressure (water density) increases. However, high water densities increase the selectivity of the dehydrogenation reaction.

There has also been a continued interest in developing chemical catalysts to produce acrylic acid from lactic acid (Fan et al 2009). For the first time Holman (U.S. Pat. No. 2,859,240) showed the vapor phase dehydration of lactic acid and lactic acid ester leading to the formation of acrylic acid and acrylic acid ester respectively. This vapor phase dehydration reaction involved catalysts consisting of the sulfates and phosphates of metals of groups I and II. This catalytic dehydration reaction was conducted within the temperature range of 200° C. to 600° C. with the conversion rate in the range of 9% to 23% depending on the composition of the catalyst used.

U.S. Pat. No. 4,729,978 discloses a process for producing an acidic dehydration catalyst suitable for the dehydration of lactic acid to acrylic acid. In the preparation of the catalysts, metal oxide carrier selected from the group consisting of silica, titanium, and aluminum is impregnated with phosphate salt. The impregnated carrier was further buffered with a base in order to improve the selectivity of the catalyst for acrylic acid production while decreasing the level of undesirable products such as acetaldehyde.

U.S. Pat. No. 4,786,756 discloses an aluminum phosphate catalyst for the vapor phase conversion of lactic acid or ammonium lactate solution into acrylic acid. The acrylic acid yield was 43.3% and 61.1% with lactic acid and ammonium lactate respectively as the reactant. The aluminum phosphate catalyst was pre treated with an aqueous inorganic base before its use in the vapor phase conversion of the lactic acid and ammonium lactate to acrylic acid. The pretreatment of the catalyst with an aqueous inorganic base increased the selectivity of the reaction to acrylic acid. The presence of water in the feed in the form of steam was also found to increase the selectivity.

U.S. Pat. Nos. 5,071,754 and 5,252,473 disclose a process for converting methyl lactate into methyl acrylate in the vapor phase using crystalline hydrated and partially calcined calcium sulfate as a catalyst. In this reaction, 15% by weight powdered calcium metaphosphate was added as promoter. There was 50% methyl lactate conversion accompanied by the production of 5 to 14% methyl acrylate and 5 to 19% acrylic acid production in the resulting liquid product.

In recent years efforts have been made to avoid the formation of self-reaction product lactide. The presence of hydroxyl group within the lactic acid molecule results in the formation of lactide due to its interaction with the carboxyl group of another lactic acid molecule. U.S. Pat. No. 6,545,175 discloses the esterification of hydroxyl group at the alpha position in methyl lactate. Methyl lactate was reacted with succinic acid anhydride in the presence of sulfuric acid at 70° C. resulting in the formation of succinic acid (ethyl-1-methoxycarbonyl) ester with 98% yield. Similarly, U.S. Pat. No. 6,992,209 discloses the formation of methyl α-acetoxy propionic ester and 2-acetoxy propionic acid in the ratio of 1:1 by means of reacting methyl lactate with glacial acetic acid and sulfuric acid at 73° C. The combined acid and methyl ester product yield was approximately 95% of theoretical estimate. It has been proposed that the thermal decomposition of succinic acid (ethyl-1-methoxycarbonyl) ester, methyl α-acetoxy propionic ester and 2-acetoxy propionic acid would yield pure acrylic acid and acrylic ester without contaminating lactide and further degradation products resulting from the decomposition of lactide. However, the efficiency of conversion of methyl lactate to acrylic acid ester and acrylic acid through this intermediary compounds remains to be established.

U.S. Pat. No. 7,538,247 discloses a process for preparation of acrylic acid, acrylic acid esters, and acrylic amide from α- or β-hydroxycarboxylic acids. The vapor phase process for the conversion of α- or β-hydroxycarboxylic acid to acrylic acid, acrylic acid esters, and acrylic amide was carried out in the temperature range of 250° C. to 300° C. Disclosed in this US patent is the conversion of the primary reactant into desirable product in the range of 83% to 97%.

U.S. Pat. No. 7,687,661 provides a process for conversion of salts of β-hydroxy carbonyl compounds into α,β-unsaturated carbonyl compounds and/or salts of α,β-unsaturated carbonyl compounds.

So far all efforts to determine the conditions for the dehydration of lactic acid and 3-hydroxypropionic acid have been carried out with pure source materials derived from petrochemical feedstock and there is a need in the field for identifying the conditions and catalysts for the conversion of lactic acid and its salt present in the fermentation broth into acrylic acid and acrylic acid esters without the need for purifying the lactic acid from the fermentation broth to very high levels of purity.

### SUMMARY OF THE INVENTION

This invention discloses the process for preparing acrylic acid and acrylic acid esters through dehydration and esterification reactions from lactic acid derived from renewable resources through biological fermentation. The biological fermentation required for the practice of this present invention involves robust biological catalysts with the capacity for using renewable resources in the production of hydroxy propionic acids such as alpha-hydroxy propionic acid or beta-hydroxy propionic acid.

In one embodiment, the present invention provides a process for manufacturing acrylic acid from the alpha-hydroxy carboxylic acid namely lactic acid and its derivatives obtained from the fermentation broth. The list of lactic acid derivatives suitable for the present invention includes inorganic salts of lactic acid, lactic acid dimer, lactic acid oligomer, and alkyl esters of lactic acid wherein the alkyl group is derived from C1-C10 alkyl alcohol. The term C1-C10 alkyl alcohol refers to the alcohols in which the alkyl group has one to ten carbon atoms. The list of salts of lactic acid includes sodium, ammonium, potassium, and calcium salts of lactic acid. The lactic acid suitable for the present invention can be in the D, (−) isomeric form, L, (+) isomeric form, or in the dimeric or oligomeric form derived from D, (−) isomeric form and L, (+) form of lactic acid. A racemic mixture of D, (−) and L, (+) isomeric forms of lactic acid is also suitable for the present invention.

In one aspect of the invention, the fermentation broth useful for acrylic acid manufacturing is derived from the cultures of the bacterial species including Escherichia coli and Bacillus coagulans selected for lactic acid production in a commercial scale. In another aspect of the present invention, the fermentation broth is derived from the culture fluid of the filamentous fungal species selected for lactic acid production. In yet another aspect of the present invention, the fermentation broth is derived from yeast species known to produce lactic acid in industrial scale.

In one embodiment of the present invention, the fermentation broth is subjected to one or more process steps including filtration, acidification, crystallization, pervaporation, electrodialysis, ion exchange, liquid-liquid extraction, and simulated moving bed chromatography to enrich the lactic acid content and to remove the impurities from the fermentation broth. In one aspect, the lactic acid enriched fraction is subjected to an esterification reaction with a C1-C10 alkyl alcohol in the presence of an esterification catalyst. The resulting lactic acid ester is subjected to a vapor phase dehydration reaction in the presence of a dehydration catalyst leading to the formation of corresponding acrylic acid ester. In another aspect of the present invention, the lactic acid enriched fraction obtained from fermentation broth through one or other purification processes is subjected to vapor phase dehydration reaction in the presence of a dehydration catalyst to yield acrylic acid. The acrylic acid resulting from the dehydration reaction is subsequently subjected to esterification reaction in the presence of an esterification catalyst and a C1-C10 alkyl alcohol to produce acrylate ester.

In another embodiment of the present invention, the fermentation broth comprising ammonium lactate is subjected to heat treatment to release ammonia from ammonium lactate leading the accumulation of lactic acid and its dimer known as lactide. The lactide thus formed is subjected to esterification reaction in the presence of an alcohol and an esterification catalyst. The lactic acid ester thus formed is subsequently subjected to vapor phase dehydration reaction in the presence of a dehydration catalyst leading to the production of corresponding acrylic acid ester. The catalysts suitable for the dehydration of lactic acid ester at the elevated temperature include solid acid catalysts, base catalyst, and metal oxides. In one aspect of the present invention, the catalysts suitable for dehydration of lactic acid are molecular sieve catalysts including various forms of zeolites.

In yet another most preferred embodiment of the present invention, the fermentation broth containing ammonium lactate is concentrated and subjected to esterification reaction with a C1-C10 alkyl alcohol. In the preferred aspect of the present invention, the esterification reaction is carried out in the absence of any exogenous esterification catalyst. The ammonia released during the heat-induced concentration process is captured through condensation reaction for recycling. Further ammonia release occurs during the esterification reaction at elevated temperature and atmospheric pressure. The ammonia thus released during the esterification reaction is driven out of the esterification reaction vessel by a stream of inert gas and captured for recycling in the fermentation process. The lactic acid ester obtained in the first stage is subsequently subjected to dehydration reaction to produce a corresponding acrylic acid ester.

### BRIEF DESCRIPTION OF THE DRAWINGS

FIG. 1 Process flow diagram for manufacturing acrylic acid and acrylic acid esters from fermentation broth containing ammonium lactate. Shown in this figure are four different pathways through which acrylic acid ester can be manufactured starting with the fermentation broth containing ammonium lactate. In one pathway, lactic acid is purified from the fermentation broth using a variety of technologies known in the field such as microfiltration, ultrafiltration, acidification, crystallization, chromatography, electrodialysis, and ion exchange. The highly purified lactic acid is subjected to vapor phase dehydration reaction at elevated temperatures in the presence of appropriate catalyst to produce acrylic acid which in turn is subjected to esterification reaction in the presence of an esterification catalyst to produce acrylic acid ester. The second pathway involves a dehydration of lactic acid in the fermentation broth without much purification followed by an esterification reaction to produce acrylic acid ester. In the third pathway, the ammonium lactate in the fermentation broth is subjected to simultaneous dehydration and esterification reactions using appropriate catalysts to produce acrylic acid ester. In the fourth pathway, ammonium lactate in the fermentation broth without much purification is subjected to esterification reaction first to produce a lactic acid ester which in turn is subjected to dehydration reaction in the presence of a dehydration catalyst. In the fourth pathway for producing acrylic acid ester, the first esterification reaction is preferentially carried out in the absence of any exogenous esterification catalyst.

FIG. 2 Process flow diagram for conversion of lactic acid to acrylic ester through esterification reaction followed by dehydration reaction.

FIG. 3 Process flow diagram for the conversion of lactic acid to acrylic acid ester through dehydration reaction followed by esterification reaction.

FIG. 4 Process flow diagram for the conversion of lactide to acrylic acid ester through esterification reaction followed by dehydration reaction.

FIG. 5. Process flow diagram for the conversion of ammonium lactate to acrylic acid ester through esterification reaction followed by dehydration reaction.

FIG. 6 Kinetics of the production of lactic acid (g/L) during anaerobic fermentation of glucose with TG160 strain of E. coli. Lactic acid production reached a maximum of about 75 g/L at 22 hours after the start of the fermentation.

FIG. 7 A typical gas chromatographic profile for a calibrating standard mixture of starting solution and the reaction products.

FIG. 8 Configuration of fixed bed reactor system used in testing the efficiency of dehydration catalysts.

FIG. 9 Kinetics of butyl lactate formation in an esterification reaction using chemically pure dimer of lactic acid (lactide) and butyl alcohol as the starting materials. Also included in the esterification reaction mixture was the amberlyst resin as an esterification catalyst. The catalyst was used at two different concentrations (2.8 wt % and 5.6 wt %).

FIG. 10 Kinetics of butyl lactate formation in an esterification reaction using fermentation broth containing ammonium lactate and butyl alcohol. The esterification was carried out at an elevated temperature under atmospheric pressure in the absence of any exogenous esterification catalysts.

FIG. 11 A typical gas chromatographic profile for an esterification reaction done using fermentation broth containing ammonium lactate and butyl alcohol.

### DETAILED DESCRIPTION OF THE PREFERRED EMBODIMENTS

The present invention provides a process for the production of α,β-unsaturated organic acids and its derivatives from α-hydroxy carboxylic acid or β-hydroxy carboxylic acid and their derivatives obtained from biological feedstock through fermentation. More specifically the present invention describes catalysts and the conditions useful in the conversion of lactic acid (α-hydroxy propionic acid) and lactic acid esters into acrylic acid and acrylic acid esters respectively. Also provided in this present invention are the catalyst and the conditions for the esterification the acrylic acid (α-β unsaturated propionic acid) derived from the dehydration of lactic acid. The dehydration and esterification reactions are conducted in a reactor vessel maintained at an elevated temperature and atmospheric pressure. Although the findings of this present invention have only been exemplified with the α-hydroxy propionic acid, the teaching of this present invention can readily be applied to the conversion of β-hydroxy carboxylic acid and its derivatives into acrylic acid and acrylic acid esters.

The term “lactic acid” as used herein refers to 2-hydroxy propionic acid also known as α-hydroxy propionic acid and includes lactic acid monomer. The term “lactic acid derivatives” as used herein refers to, but not limited to, lactic acid dimmers (lactide), lactic acid trimers, low molecular weight polymers of lactic acid, salts of lactic acid and alkyl lactate. Lactide is also known as dilactide and is derived from the condensation of two molecules of lactic acid in a dehydration reaction. Alkyl lactate is derived from the condensation of lactic acid with alcohol. The alcohol suitable for the formation of alkyl lactate of the present invention is a member of alkanol (C1 to C10), a group of alkyl alcohols with 1 to 10 carbon atoms. During the process for fermentative production of lactic acid, alkali materials are added in order to maintain the pH of the fermentation medium leading to the accumulation of lactic acid in the form of salt in the fermentation medium. For example, when ammonium hydroxide is used to maintain the pH of the fermentation medium, lactic acid accumulates in the fermentation medium as ammonium lactate which is referred herein as a lactic acid derivative. The pH of the fermentation medium for the production of lactic acid can also be controlled with the addition of other alkali materials such as Ca(OH)₂, NaOH, and KOH leading to the formation of lactic acid derivatives such as calcium lactate, sodium lactate and potassium lactate.

The term “acrylic acid” as used herein refers to α-β unsaturated propionic acid derived from the dehydration reaction involving either α-hydroxy propionic acid or β-hydroxy propionic acid. The term “acrylic acid derivatives” as used herein refers to alkyl acrylate derived either from the condensation of acrylic acid with an alcohol selected from alkanol (C1-C10 alcohol) or from the dehydration of an alkyl lactate.

The term “esterification” or “esterification reaction” as used herein refers to the condensation of acid and alcohol molecules.

The term “dehydration” or “dehydration reaction” as used herein refers to the removal of a water molecule from an acid or an ester molecule.

The term “catalyst” as used herein refers to a chemical entity which is used to lower the activation energy for a chemical reaction leading to an increase in the rate of the chemical reaction. The term “exogenous catalyst” as used herein refers to the chemical entity which is added to any chemical reaction from outside source in order to lower the activation energy required for chemical reaction and to improve the overall rate of the chemical reaction. This term “exogenous catalyst” is used to distinguish the situation wherein some of the substrates of the chemical reaction itself can act as a catalyst. In the present invention, catalysts are used to improve the rate of either esterification reaction or dehydration reaction.

The term “source material” as used herein refers to the material fed into the reactor vessel in order to initiate a chemical conversion reaction. This term encompasses the lactic acid and all of its derivatives obtained from fermentation broth and introduced into the primary reaction vessel as the substrate for dehydration reaction. In certain manufacturing process the products from the primary reaction vessel comprising primarily acrylic acid would be used as the source material for esterification reaction in the secondary vessel. In yet other manufacturing process, an esterification reaction occurs in the primary reaction vessel using lactic acid or lactic acid derivative as the source material leading to the production of lactic acid ester. The lactic acid ester thus formed in the primary reaction vessel would become the source material for the dehydration reaction occurring in the secondary reaction vessel.

The term “conversion” as used herein refers to the quantity of a source material consumed in a specific reaction and is provided as the percentage of moles of source material consumed with reference to the moles of source material supplied.

The term “conversion products” as used herein includes all of the products derived from the source material within the reaction vessel. This would include the desirable product as well as the byproducts derived from the degradation of the reactants and the primary products.

The term “molar selectivity” or “selectivity” as used herein refers to the quantity of a particular conversion product with reference to the quantity of the source material consumed and is provided as a ratio of moles of product formed to the moles of source material consumed.

The term “molar yield” as used herein refers to the moles of the product formed to the moles of source material fed to the reaction vessel.

The term “calcined” as used herein refers to the high temperature treatment of catalyst in order to reduce the water content of the catalysts significantly.

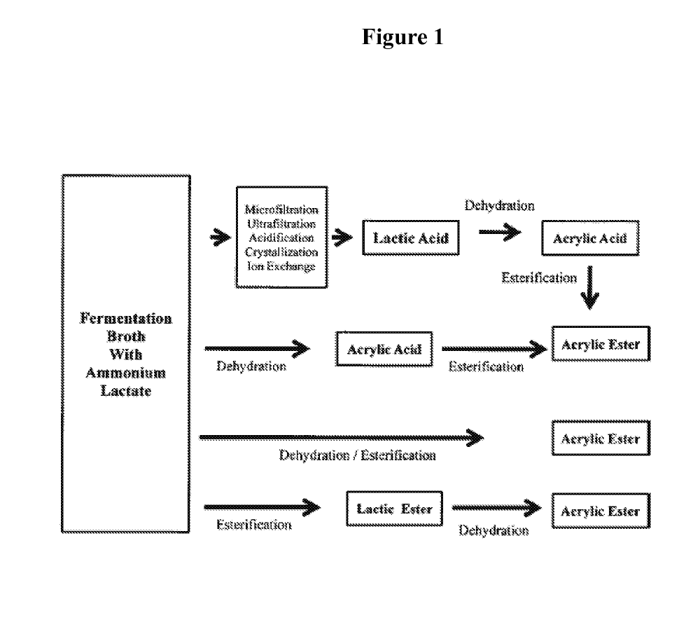

As illustrated in FIG. 1, this present invention provides four different routes for the production of acrylic acid and acrylic acid ester from fermentation broth containing ammonium lactate. The processes described in this present invention involves two primary reactions namely dehydration reaction and esterification reaction. Both these reactions can be carried out either in the aqueous phase or in the vapor phase. The reactions occurring in the vapor phase are preferred. The vapor phase reaction can be carried out in a batch, fed-batch or continuous mode.

In one embodiment of the present invention, lactic acid is derived from the lactic acid salts such as ammonium lactate present in the fermentation broth using a process involving microfiltration, ultrafiltration, acidification, crystallization, chromatography, electrodialysis and ion exchange steps. The lactic acid thus produced is subjected to dehydration reaction to yield acrylic acid which can subsequently be esterified to yield acrylic acid ester. In another embodiment of the present invention, the lactic acid salt present in the fermentation broth such as ammonium lactate is subjected to dehydration reaction to yield acrylic acid which can be subjected to esterification reaction to produce acrylic acid ester. Alternately, ammonium lactate may first be subjected to esterification reaction followed by dehydration reaction involving lactic acid ester formed in the first step. In yet another embodiment of the present invention, the lactic acid salts such as ammonium lactate present in the fermentation broth is subjected to simultaneous dehydration/esterification reaction to yield acrylic acid ester. In yet another embodiment of the present invention, the lactic acid obtained from the fermentation broth is subjected to esterification reaction to yield lactic acid ester which is subsequently dehydrated to yield acrylic acid ester. The acrylic acid ester thus obtained through one or more of the processes described above is subjected to ester hydrolysis reaction to produce acrylic acid in high levels of purity and recover the alcohol originally used to produce lactic acid ester. The alcohol thus recovered from acrylic acid ester hydrolysis reaction can be recycled.

The esterification and dehydration reactions are carried out either in the absence or presence of a chemical catalyst. Under certain circumstances as illustrated below with the examples, it is possible to carry out the esterification reaction in the absence of any exogenous catalysts. The esterification reaction in the absence of any exogenous catalyst is preferred.

The catalysts are selected without limitation based on their ability to improve the overall conversion efficiency of the chemical reaction and the selectivity for a particular end product. It is preferred that the dehydration and esterification reactions be done in the vapor phase over heated catalysts in a continuous mode.

Dehydration catalysts for the present invention include but not limited to solid oxides, zeolites, solid acids, acidic catalysts, weakly acidic catalysts, strongly acidic catalysts, basic catalysts, ion exchange resins, and acidic gases. These various catalysts can be used alone or in any suitable combinations.

The list of solid oxide catalyst suitable for the present invention includes but not limited to TiO₂, ZrO₂, Al₂O₃, SiO₂, ZnO₂, SnO₂, WO₃, MnO₂, Fe₂O₃, V₂O₅, SiO₂/Al₂O₃, ZrO₂/WO₃, ZrO₂/WO₃, ZrO₂/Fe₂O₃, ZrO₂/MnO₂ or combinations thereof.

Zeolites are the aluminosilicate members of the family of microporous solids known as “molecular sieves.” In the broadest sense, any material that can exclude molecular species by size can be considered a molecular sieve. The diameter of pores in the zeolite catalyst may be in the range of 1 to 20 angstroms. The preferred pore size in the zeolite catalyst is in the range of 5 to 10 angstroms. The zeolite mediated catalysis takes place preferentially within the intracrystalline void. Catalytic reactions are affected by aperture size and types of channel system, through which reactants and products must diffuse.

The zeolite catalyst may be derived from naturally occurring materials or may be chemically synthesized. The zeolite framework is made up of SiO₄ tetrahedral linked together by sharing of oxygen ions. Substitution of Al³⁺ for Si⁴⁺ generates a charge imbalance, necessitating the inclusion of a cation such as K⁺ Na⁺, and Cu⁺⁺. The structures contain channels or interconnected voids that are occupied by the cations and water molecules. Zeolites have a general molecular formula M_{x/n}[(AlO₂)ₓ(SiO₂)_{y}].zH₂O where n is the charge of the metal cation, M. M is usually Na⁺, K⁺ or Ca²⁺ and z is the number of moles of water of hydration which is highly variable. An example mineral formula is: Na₂Al₂Si₃O₁₀.2H₂O, the formula for natrolite.

Al₂O₃ and zeolite with high surface area are the most preferred dehydration catalyst. The dehydration catalysts suitable for the present invention have a surface area in the range of 100 m²/g to about 500 m²/g. The preferred surface area of the catalyst suitable for the present invention is at least about 125 m²/g and the most preferred surface area of the catalyst for the present invention is at least 150 m²/g.

The dehydration reaction of the present invention can also be conducted in the absence any catalyst enumerated above and only in the presence of inert solid support such as glass, ceramic, porcelain, or metallic material present within the reaction vessel.

The aluminum silicate compounds may function both as an esterification and dehydration catalyst. Thus when a fermentation broth containing α-hydroxy propionic acid is used as the source material, in the first step of the catalysis, the aluminum silicate catalysts would catalyze the removal of water molecule from the α-hydroxy propionic acid leading to the formation of α-β-unsaturated propionic acid. In the second stage, the same aluminum silicate catalyst would catalyze the formation of an ester bond between the carboxyl group of α-β unsaturated propionic acid and an alcohol.

The acidic catalysts useful in the present invention can either be a liquid catalyst or solid catalyst. The liquid acidic catalysts include sulfuric acid, hydrogen fluoride, phosphoric acid, and paratoluene sulfonic acid. The solid acid catalysts are preferred over the liquid acid catalyst. This is particularly important when there is a need to separate the catalyst from the waste before disposal. In general the solid catalyst is obtained by contacting a hydroxide or hydrated hydroxide of a metal belonging to group IV of the periodic Table with a solution containing a sulfurous component and calcining the mixture at 350 to 800° C. The solid acid catalyst has an acidity higher than that of 100% sulfuric acid. Because of their high acidity, the solid acid catalysts exhibit high catalyzing power in various acid-catalyzed reactions. In addition the solid catalysts have certain other advantageous features. They show low corrosiveness; they can be separated easily from the reactants; they do not require disposal of waste acids, and can be reused. For these reasons, the solid acid catalysts are expected to be substituted for conventional acid catalysts.

Acidic or weakly acidic catalysts suitable for the present invention include titania catalysts, SiO₂/H₃PO₄ catalysts, fluorinated Al₂O₃ (e.g., Al₂O₃.HF catalysts, Nb₂O₃/SO₄⁻² catalysts, Nb₂O₅.H₂O catalysts, phosphotungstic acid catalysts, phosphomolybdic catalyst, sililcomolybdic acid catalysts, silicotungstic acid catalysts, acidic polyvinylpyridine hydrocholoride catalysts, hydrated acidic silica catalysts, and combination thereof.

Even when dealing with a single solid catalyst several changes in the catalyst composition can be made with a goal of improving the conversion rate and selectivity of the catalyst. For example, while using sodium phosphate catalyst supported on silica a number changes can be made to the composition of the catalyst in order to improve the selectivity for a particular end product. The parameter that can be altered to improve the selectivity of the catalyst include NaH₂PO₄ loading, and Na₂O/P₂O₅ ratio. An optimal loading of NaH₂PO₄ in the range of 1.0 to 2.1 mmol g⁻¹ is preferred. Similarly, catalyst with Na₂O to P₂O₅ ratio in the range of 0.77 to 2.0 is preferred. This range of Na₂O to P₂O₅ can be obtained by the addition of either H₃PO₄ or Na₂CO₃ to the aqueous solution containing NaH₂PO₄.

In the case of NaY zeolite catalysts, it is possible to improve the conversion efficiency and selectivity by means of modifying the catalyst with potassium or rare earth metals including lanthanum, cerium, samarium and europium. Similarly, in the case of calcium sulfate catalysts, one can improve the performance of the catalysts in terms of conversion and selectivity either by using different carrier gas, or by controlling the temperature for calcining the catalyst, or by controlling the feed concentration and feed rate or the duration of the contact with the catalyst.

A preferred titania catalyst is Ti-0720® (Engelhard, Iselin, N.J., USA). A preferred polyvinylpyridine hydrochloride catalyst is PVPH⁺Cl⁻® (Reilly, Indianapolis, Ind., USA). A preferred hydrated acidic silica catalyst is ECS-3® (Engelhard, Iselin, N.J., USA).

Basic catalysts suitable for the present invention include, but are not limited ammonia, polyvinylpyridine, metal hydroxide, Zr(OH)₄, and amine with the general formula NR1R2R3, where R1, R2, and R3 are independently selected from the group of side chain or functional groups including, but not limited to e.g., H, hydrocarbons containing from 1 to 20 carbon atoms, alkyl and/or aryl groups containing from 1 to 20 carbon atoms, or combinations thereof. When ammonium lactate is used as the source material for acrylic acid production and subjected to high temperature treatment, it decomposes with the release of ammonia and lactic acid. The ammonia thus released from the decomposition could act as a catalyst for the dehydration of lactate.

In the first stage of the present invention, lactic acid is manufactured from biological feedstock in commercially significant quantities using microorganisms. In the second stage of the present invention, the lactic acid and its derivatives recovered from the biological fermentation in a cost effective manner are subjected to catalytic dehydrogenation reaction for the purpose of producing acrylic acid and its derivatives. Preferably, the catalytic dehydrogenation reaction can be carried out with the crude fermentation broth comprising lactic acid.

The fermentation process for producing lactic acid can either be conducted in a batch mode or in a continuous mode. A large number of carbohydrate materials derived from natural resources can be used as a feedstock for the fermentative production of lactic acid. Sucrose from cane and beet, glucose, whey containing lactose, maltose and dextrose from hydrolyzed starch and glycerol from biodiesel industry are suitable for the fermentative production of lactic acid. Microorganisms can also be created with the ability to use pentose sugars derived from hydrolysis of cellulosic biomass in the production of lactic acid. A microorganism with ability to utilize both 6-carbon containing sugars such as glucose and 5-carbon containing sugars such as xylose simultaneously in the production of lactic acid is a highly preferred biocatalyst in the fermentative production of lactic acid. Hydrolysate derived from cheaply available cellulosic material contains both C-5 carbon and C-6 carbon containing sugars and a biocatalyst capable of utilizing simultaneously C-5 and C-6 carbon containing sugars in the production of lactic acid is highly preferred from the point of producing low-cost lactic acid suitable for the conversion into acrylic acid and acrylic acid ester.

Acid-tolerant homolactic acid bacteria is suitable for the present invention. By “homolactic” it is meant that the bacteria strain produces substantially only lactic acid as the fermentation product. The acid-tolerant homolactic bacteria is typically isolated from the corn steep water of a commercial corn milling facility. An acid tolerant microorganism which can also grow at elevated temperatures is preferred. The microorganism which produces at least 50 g of lactic acid per liter of the fermentation fluid is favored. In terms of productivity, a fermentation run which yields 4 grams of lactic acid per liter per hour is desirable.

The list of the microorganisms well known for the production of lactic acid in commercial scale includes Escherichia coli, Bacillus coagulans, Lactobacillus delbruckii, L. bulgaricus, L. thermophilus, L. leichmanni, L. casei, L. fermentii, Streptococcus thermophilus, S. lactis, S. faecalils, Pediococcus sp, Leuconostoc sp, Bifidobacterium sp, Rhizopus oryzae and a number of species of yeasts in industrial use.

Recently granted U.S. Pat. No. 7,629,162, which is incorporated herein by reference, discloses derivatives of ethanolgenic Escherichia coli KO11 strain constructed for the production of lactic acid. The lactic acid producing strains disclosed in this US patent were obtained by deleting the genes that encode competing pathways followed by a growth-based selection for mutants with improved performance. These transformed E. coli are useful for providing increased supply of lactic acid for use in industrial applications.

Lactic acid may exist as either of two stereochemical enantiomers or so-called “optical isomers” namely D, (−)-lactic acid and L, (+)-lactic acid. A mixture of 99% “optical” purity is either (a) 99% D and 1% L or (b) 1% D and 99% L. A mixture of molecules of both forms is called a racemic mixture, or DL-lactic acid. The optical purity refers to the optical purity of the mixture of all forms of lactate, lactic acid, monomers, dimers etc. Salts of lactic acid also retain optical purity, as do compounds produced by chemical reaction of lactic acid, depending on the reaction and purification sequence.

Lactic acid and its derivatives obtained from the biological fermentation broth are preferable in practicing the present invention. The fermentation broth contains about 6-15% lactic acid on weight/weight (w/w) basis and it is necessary to recover the lactic acid in a concentrated form. The recovery of lactic acid in a concentrated form from fermentation broth can be carried out in one of the known methods in the art. Several different methods are known in the art for recovering lactic acid from fermentation broth. Any one of those known methods or combination of several methods can be followed to obtain lactic acid from the fermentation broth in a concentrated form suitable for the use in the preparation of acrylic acid and acrylic acid ester by using the processes disclosed in this present invention.

During the industrial production of lactic acid, at least one alkali material such as NaOH, CaCO₃, (NH₄)₂CO₃. NH₄HCO₃ and NH₄OH is added to the fermentation broth in order to maintain the near neutral pH of the growth medium. Addition of alkali to the fermentation broth results in the accumulation of lactic acid in the form of inorganic salts. Ammonium hydroxide is the preferred alkali material for maintaining the neutral pH of the fermentation broth. With the addition of ammonium hydroxide to the fermentation medium, ammonium lactate accumulates in the fermentation broth. Ammonium lactate has higher solubility in aqueous solution and therefore it is possible to increase the concentration of ammonium lactate in the fermentation broth.

One way to obtain lactic acid from the fermentation broth containing ammonium lactate is to subject the fermentation broth to micro and ultra filtration followed by ion exchange chromatography. The sample coming out of ion exchange chromatography is subjected to conventional electrodialysis to obtain lactic acid in the form of concentrated free acid.

Another method for recovering lactic acid from fermentation broth is to use the acidification and crystallization procedures. For example, when the fermentation is carried out in the presence of calcium carbonate, it is possible to recover the lactic acid by acidification with sulfuric acid. This results in the precipitation of calcium sulfate, while free lactic acid remains in the mother liquor. Subsequently, free lactic acid present in the mother liquor is extracted with a suitable organic extractant to yield an extract which is back-extracted with water to recover free lactic acid in a concentrated form. The long-chain trialkyl amines such as triethylamine, tridodecylamine, triisooctylamine, tricaprylylamine and tridodecylamine are useful as extractants in the recovery of free lactic acid. The term amine salt or amine lactate refers to the species formed when lactic acid is extracted into the amine extractant phase.

The extraction power of an amine-containing organic extractant is enhanced by the incorporation of a non-carboxylic, neutral polar organic compound, e.g. an alkanol such as n-butanol, a ketone such as butanone, an ester such as butylacetate, an ether such as dibutylether, and a bifunctional compound such as CH₃CH₂CH₂OHCH₂CH₂OH. Such compounds, generally referred to as enhancers, modifiers or active diluents, increase the base strength of the amine in the extractant and thereby facilitate the transfer of carboxylic acid from the starting aqueous solution such as a fermentation broth, into the organic extractant phase. The presence of an extraction enhancer shifts the carboxylic acid equilibrium in an aqueous phase/organic extractant phase system in favor of the organic phase. This very shifting of equilibrium, however, creates a problem for the back extraction in that transfer of the carboxylic acid from the organic-to the aqueous phase is inhibited. In fact, this inhibition may be so pronounced as to render back extraction of the organic acid with water impractical even at temperature close to 100° C. Several approaches have been proposed to overcome this difficulty inherent in carboxylic acid recovery processes of this kind. According to one approach, back extraction is foregone altogether and carboxylic acid is recovered from the organic extract by distillation. By another approach, back extraction is carried out above the later boiling temperature so as to increase the degree of hydrolysis of the amine-carboxyl complex and thereby provide for an acceptable rate of back extraction.

The lactic acid from the fermentation broth can also be directly recovered by adsorbing onto a solid-phase polymer containing tertiary amine. After the polymer is saturated, it is preferably water washed and the adsorbed lactic acid can be recovered using a suitable agent. Suitable desorbing agent includes polar organic solvents methanol as well as hot water. After elution from the column, the lactic acid can be concentrated by evaporation, distillation, or any other suitable means known in the art.

Alternately, calcium lactate is reacted with a source of ammonium ions, such as ammonium carbonate or a mixture of ammonia and carbon dioxide, thereby producing an ammonium lactate. Contaminating cations can be removed by ion exchange. The free lactic acid can be separated from the ammonium ions, preferably by salt-splitting electrodialysis.

In yet another embodiment of the present invention, the acidified fermentation broth containing lactic acid is passed over a cation exchange resin to give a fraction that has maximum of 25% lactic acid salts relative to the dry weight of the solution. The fraction eluted from the ion exchange column is subjected to bipolar fractionating-electrodialysis. The resulting lactic acid is further purified, concentrated and the recovered.

It is also desirable to use simulated moving bed chromatography, precipitation, crystallization and evaporation procedures to recover more than 90% of lactic acid from the fermentation broth.

Lactic acid obtained from the fermentation broth can be subjected to esterification process to recover lactic acid ester as an end product. A variety of alcohols including, but not limited to methyl alcohol, ethyl alcohol, isopropyl alcohol, n-propyl alcohol, isobutyl alcohol, n-butyl alcohol, sec-butyl alcohol, 2-ethyl hexanol, isononanol, isodecylalcohol, and 3-propyl heptanol can be used in the esterification reaction. Butanol obtained from fermentative process is a preferred alcohol. The commercial production of butanol through fermentative process is now well established as evidenced by a number of recently issued United States patents (U.S. Pat. No. 7,851,188; U.S. Pat. No. 7,910,342 and U.S. Pat. No. 7,993,889) and United States Patent Application Publications (US2011/0159,558; US2011/0195,505; US2011/0112,334; US2010/0129,886; US2010/0221,801; US2010/0062,505 and US2011/0183,392).

Esterification can occur between two lactic acid molecules leading to the formation of lactide. In addition, the oligomers of lactate ester can originate from the esterification of the oligomers of lactic acid. It is preferable to use lactate esters which is free of lactide and oligomers of lactate esters.

The molar ratio between lactic acid and esterifying alcohol and the temperature, and the pressure of the reaction vessel are crucial in achieving the desirable efficiency of esterification reaction. For example, the conditions for a continuous process for the preparation of ethyl lactate require that molar ratio of at least 2.5 exists between ethanol and lactic acid. The preferred range for the molar ratio between ethanol and lactic acid is in the range of 3 to 4. The esterification reaction is conducted at 100° C., and under pressure ranging from 1.5 to 3 bar and preferably ranging from 1.5 to 1.8 bar.

The esterification reaction is carried out in the presence of an acid catalyst which is soluble or insoluble in the esterification reaction medium. The catalysts which can be used according to the present invention include 98% H₂SO₄, H₃PO₄ or methanesulfonic acid. The catalysts are used in the concentration ranging from 0.1% to 4%, and preferably in the range of 0.2% to 3%, with respect to the 100% lactic acid employed. The esterification reaction can be conducted either in a stirred reactor or in a fixed bed reactor. When the fixed bed reactor is used, solid catalysts, such as ion-exchange resins of the Amberlyst 15 type is used and the esterification is conducted by reactive distillation. The term “reactive distillation” refers to the combination of a chemical reaction and the separation of substance by distillation. The reactive distillation methods suitable for the producing a hydroxyacid ester like lactic acid ester are well known in the art. A special advantage of reactive distillation is the fact that during the esterification the water of reaction which forms is immediately removed by distillation, and therefore the reaction equilibrium is shifted in the direction of the ester formation.

In another embodiment of the present invention, methyl lactate in high yield can be obtained from esterification of aqueous crude lactic acid solution produced by sugar cane juice fermentation broth with methanol in continuous counter current trickle phase approach or in a continuous counter current bubble column.

The fermentation broth containing ammonium lactate can also be used as source of lactic acid and the alkyl lactate. The fermentation broth containing ammonium lactate is mixed with alcohol and supplied to a first reaction vessel along with an elevated heat stream comprising inert gas, alcohol vapor, carbon dioxide, or mixture of any two or more thereof. At the elevated temperature prevailing within the first reaction vessel, the ammonium lactate is decomposed into ammonia and free lactic acid. The liquid stream coming out of the first vessel would have alcohol and organic acid. The ammonia thus liberated would go out in a vapor stream out of the first vessel. When the first vessel is supplied with a catalyst for the esterification of lactic acid, the liquid stream coming out of the first vessel would also have alkyl ester of lactic acid besides alcohol and lactic acid. The liquid stream from the first reaction vessel is connected to the second reaction vessel. By means of the controlling the pressure parameters of the first vessel to favor the esterification reaction and preventing the catalyst from entering the second vessel, it is possible to recover alkyl ester of the lactic acid in increasing proportion in the second vessel. The list of alcohols suitable for this process includes, but not limited to methanol, ethanol, i-propanol, n-propanol, i-butanol, t-butanol, n-butanol, 2-ethyl hexanol, isononanol, isodecylalcohol, and 3-propylheptanol.

In another embodiment of the present invention, the lactic acid is obtained from the fermentation broth comprising ammonium lactate without resorting to the use of strong acid. Instead, heated alcohol vapor is used to elevate the temperature of the fermentation broth. With the rise in the temperature, ammonia is striped off from ammonium lactate. The lactic acid thus released with the heated alcohol vapor treatment is now available for esterification reaction. The list of alcohols suitable for the esterification reaction includes but not limited to i-butanol, t-butanol, n-butanol, i-propanol, n-propanol, ethanol, and methanol are suitable in the esterification reaction. The lactic acid esters thus formed can be recovered through differential distillation and condensation procedure as described above. In a preferred embodiment, the esterification is achieved without the addition of any exogenous catalyst and the fermentation broth containing ammonium lactate is concentrated through evaporation before subjecting it to alcohol vapor for initiating the esterification. In the most preferred embodiment a stream of inert gas is passed through the esterification reaction vessel in order to drive out the ammonia being released.

In another embodiment of the present invention, the ammonium lactate present in the fermentation broth is esterified by adding alcohol and esterification catalyst to the fermentation broth and heating the resulting mixture to a temperature below 100° C. The treatment besides decomposing ammonium lactate into free lactic acid and ammonia, initiates the esterification reaction. The ammonia thus released along with the excess of water in the original fermentation broth is removed using pervaporation membranes. In the first stage, pervaporation membrane is used for dehydration and deamination purpose. In the second stage, pervaporation membrane is used to separate alkyl lactate from free lactic acid and remaining alcohol in the reaction mixture. Thus a highly pure alkyl lactate is obtained without resorting to the any high temperature treatment required in distillation process for recovery of alkyl lactate from alcohol used in the esterification reaction.

In yet another embodiment of the present invention, the alkyl esters of lactic acid can be obtained from a lactic acid source comprising dimers and high polymers through a catalyst-free esterification process with an alcohol in the presence of water at temperature in the range of 130-250° C. for 4-11 hours at a pressure of 5-25 kg/cm².

As explained above, lactic acid and a variety of lactic acid esters can be derived from renewable resources through biological fermentation. Well characterized methods are now available to recover the lactic acid from the fermentation broth and to convert it into a lactic acid ester. Both lactic acid and lactic acid ester can be subjected to high temperature catalytic dehydration reaction to produce acrylic acid and acrylic acid esters. Given below are the details about the system that can be used to manufacture acrylic acid and acrylic acid esters from the ammonium lactate containing fermentation broth. Also provided here is a description of methods that can be followed to recover acrylic acid and acrylic acid ester manufactured from ammonium lactate containing fermentation broth.

In its simplest construction, the system for conducting dehydration and esterification reactions of lactic acid and its esters comprise a reactor located within a heating source. Alternately the reactor may be in close physical contact with a heating source so that there is a uniform heat conductance from the heat source to the reactor. The reactor and the heating source are connected through a series of thermocouples. The thermocouples are spread across the length of the reactor to assure that the reactor is heated uniformly across its length by the heating source.

The reactor is filled with one or other types of catalysts. Under those conditions, where the dehydration reaction is conducted without any exogenously added catalyst, the reactor is filled with inert materials, such as glass, ceramic and brick. The reactor is maintained at atmospheric pressure and kept at temperatures above the boiling temperature of water. The container with feed source is connected to the one end of the reactor through stainless steel tubing and the feed source is fed into the reactor at a weight hourly space velocity (WHSV) optimized for the maximum conversion of the feed source within the reactor.

In certain other designs, the feed source is mixed with stabilizing agents and inhibitors of acrylic acid polymerization reaction in a mixer tank before feeding it into the reactor. Suitable stabilizing agents and inhibitors include, but are not limited to phenolic compounds such as dimethoxy phenol (DMP) or alkylated phenolic compounds such as di-tert-butyl phenol, quinines such as t-butyl hydroquinone or the monomethyl ether of hydroquinone (MEHQ), and/or metallic copper or copper acetate. In yet other designs, the feed source, catalyst and polymerization inhibitor are mixed together in a mixing tank and the mixer is fed into the reactor maintained at a temperature suitable for dehydration reaction to occur.

In yet another embodiment of the system design, the feed flow from mixer tank is taken through a spray dryer/evaporator unit before being fed into the reactor. This passage through the spray dryer/evaporator is to reduce the water content of the feed source before entering into the reactor. With the reduced water content in the feed material, the rate of catalytic conversion within the reactor is expected to increase.

The end products of the dehydration reaction occurring in the reactor are collected as an effluent stream emanating from the other end of the reactor. It is also useful to supply a gas steam to the reactor in order to drive the vapor phase along with the product of the catalytic degradation towards the effluent stream. Any required modifications can be made to this basic system design in order to accommodate any deviation in the process that may be required either to use a different feed source or to obtain a different end product. Such deviations in the system design are explained below as and when it is required.

In principle, the feed material is vaporized at appropriate temperature within the reactor and catalytic dehydration of lactic acid occurs in the vapor phase. The acrylic acid product resulting from the dehydration reaction is collected in the effluent stream emanating from the other end of the reactor. The feed source for producing acrylic acid may contain 5% to 30% lactic acid on w/w basis. Preferably the feed source containing 7.5 to 12% lactic acid on a w/w basis is fed into the reactor. It is ideal to have the lactic acid in the feed source in a monomeric form. However, with suitable catalyst, appropriate temperature and proper residence time, it is possible to breakdown the dimeric and polymeric lactic acid molecules and subject them to catalytic degradation.

It is also possible to use fermentation broth containing inorganic salts of lactic acid as a feed source. The list of inorganic salts of lactic acid suitable for the present invention includes, but not limited to ammonium lactate, sodium lactate, and calcium lactate. Fermentation broth containing ammonium lactate is the preferred feed source for dehydration reaction leading to the formation of acrylic acid. The ammonium lactate is decomposed within the reactor and the lactic acid thus released is subjected to dehydration reaction. The conversion rate and selectivity for acrylic acid production may reach as much as 95% or higher with ammonium lactate as the feed source. It is also possible to carry out the dehydration reaction of the ammonium lactate within the reactor even without any exogenously added chemical catalyst. The ammonia released from the decomposition of the ammonium lactate can act as a basic catalyst for the dehydration of lactic acid to form acrylic acid. In addition, the ammonia gas may act as a carrier gas to move the acrylic acid across the reactor towards the effluent stream.

It is also possible to carry out the dehydration and esterification reactions simultaneously in the system described above. As described above, the introduction of fermentation broth with lactic acid in the free acid, dimer or polymer forms into the reactor with appropriate chemical catalyst would result in the formation of acrylic acid as product. If suitable alcohol is also introduced into the reactor along with lactic acid in the feed source, the vapor phase esterification of the acrylic acid can be initiated. Thus with a single catalyst or multiple catalyst within the reactor, it is possible to achieve simultaneous catalytic dehydration of lactic acid and catalytic esterification of resulting acrylic acid. Depending on the source of alcohol used corresponding acrylic ester can be obtained. The list of alcohols suitable for this esterification reaction includes, but not limited to methanol, ethanol, n-propanol, isopropanol, n-butanol, isobutanol, 2-ethyl hexanol, isononanol, isodecylalcohol, and prophylheptanol.

In another embodiment of the present invention, the dehydration and esterification reactions are conducted in sequence. Following the process described above, in the first stage, lactic acid or lactide or lactic acid salt is subjected to vapor phase dehydration reaction with or without a chemical catalyst to produce acrylic acid. The resulting acrylic acid is collected in the effluent stream, mixed with appropriate alcohol and fed into a reactor with a chemical catalyst for esterification reaction. In another aspect of the present invention, the alcohol can be fed into the reactor as an independent feed stream along with the feed stream providing acrylic acid.

In yet another embodiment of the present invention, the lactic acid obtained from the fermentation broth can be esterified with a variety of alcohol to produce appropriate ester as described above. The lactic acid esters thus produced can be introduced into the reactor with dehydration catalyst maintained at appropriate temperature. The vapor phase dehydration reaction occurring within the reactor would result in the formation of acrylic acid which can be recovered as an effluent.

The composition of the source materials as well the products from dehydration and esterification reactions can be analyzed by using appropriate high performance liquid chromatography (HPLC) or gas chromatography (GC) techniques and from the data derived from HPLC or GC analysis, the conversion and selectivity value can be obtained. In developing the industrial processes, it is essential to eliminate the formation of unwanted byproducts so that the final desired product can be obtained with high level of purity.

The acrylic acid and acrylic acid esters obtained from dehydration and combined dehydration and esterification reaction can be purified using a variety of techniques well known in the art. For example the acrylic acid resulting from the dehydration reaction can be recovered through thermal distillation in the presence of polymerization inhibitor. Pervaporation process with the aid of a membrane can also be used to concentrate aqueous acrylic acid solution under mild condition without an extraction agent with low expenditure of energy. Fractional crystallization is yet another way to purify acrylic acid.

The liquid-liquid extraction process can be followed for the separation of acrylic acid from the aqueous mixture obtained at the end of the catalytic dehydration reaction involving lactic acid or its derivatives. Liquid-liquid extraction is preferred over the distillation reaction as it avoids any possible thermal degradation of acrylic acid. Liquid-liquid extraction is a diffusional separation process, wherein feed flow is brought into contact with a selected solvent. The solvent will remove acrylic acid from the rest of the components in the feed flow. The acrylic acid is subsequently recovered from the solvent stream using appropriate processes well known in the art. Several solvents including diisopropyl ether, 2-ethylhexanol, isopropyl acetate, methyl isobutyl ketone, caproic acid, enanthic acid, caprylate, methyl pelargonate, and trialkylphosphine oxide are known to be useful in extracting acrylic acid. Any one of these solvents can be used in extracting acrylic acid from an aqueous mixture.

The acrylic acid ester obtained through esterification reaction can be treated with boron trifluoride to get rid of the impurities which are detectable by discoloration after treatment of acrylic acid ester with a small amount of sulfuric acid. The acrylic acid ester obtained from esterification reaction is treated with 0.05 to 0.5% by weight, with reference to the weight of the ester, of boron trifluoride as described in the now expired U.S. Pat. No. 2,905,598 which is incorporated herein by reference. Boron trifluoride evidently forms a stable compound with the injurious impurities or reacts with them to form products of which the boiling point or decomposition temperature lie higher than the boiling point of the ester so that the ester can be separated from the impurities by simple distillation under normal or decreased pressure.

The unreacted alcohol and the acrylic acid still present in the acrylic acid ester preparation can be removed as per the process described in the now expired U.S. Pat. No. 3,157,693. According to the process in this US patent, the acrylic acid ester preparation is treated with a dilute water solution of sodium bicarbonate and subject to thermal fractionation to remove both more volatile and less volatile components present in the acrylic acid ester preparation obtained originally from the esterification reaction.

The method described in this invention for manufacturing acrylic acid and acrylic acid from renewable resources is simple. In addition, the system useful in this manufacturing process is highly configurable. Each unit operations can be adjusted in such away so that maximum conversion efficiency and selectivity can be achieved. This manufacturing process is low in cost and cause little pollution to the environment.

By strictly adhering to the above reaction conditions, one skilled in the art is able to obtain repeatedly a desired ester product in high yields substantially free from reaction complexities.

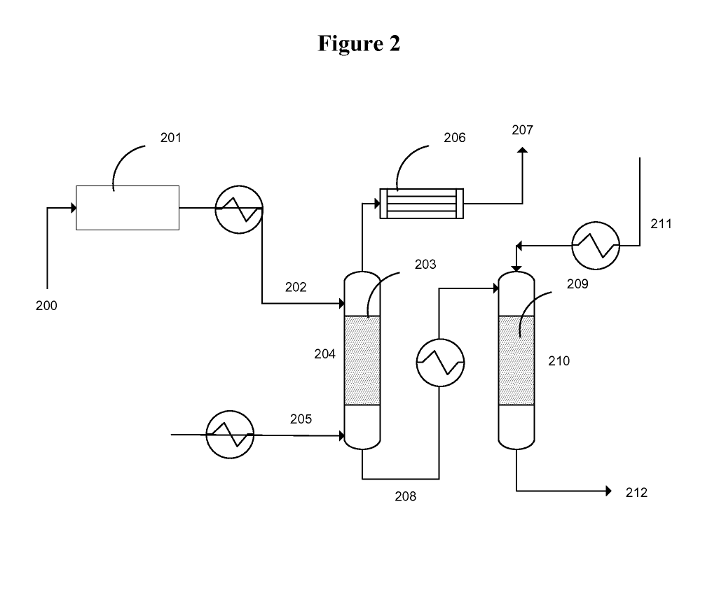

FIG. 2 provides the process flow diagram for one of the embodiments of the present invention wherein highly purified lactic acid is used as the source material. The fermentation broth containing ammonium lactate (200) is taken through a conversion process (201) to recover high purity lactic acid. The lactic acid thus recovered in high purity (202) is fed into an esterification reaction vessel (203) from the top while alcohol vapor (205) is fed from the bottom. Solid esterification catalyst (204) is kept on a solid support within the esterification vessel. The excess amount of alcohol vapor escaping the esterification vessel from the top is recovered during its passage through a condenser (206). The alcohol thus recovered (207) is recycled into the esterification vessel. Lactic acid ester and the water resulting from the esterification reaction (208) within the esterification vessel is collected at the bottom of the esterification vessel and fed as a source material on the top of the dehydration vessel (209). The dehydration vessel has solid dehydration catalyst on a solid support (210). An inert gas stream (211) is introduced form the top of the dehydration vessel to drive the acrylic acid ester to the bottom of the dehydration vessel form which point the acrylic acid ester (212) is collected and subjected to further purification.

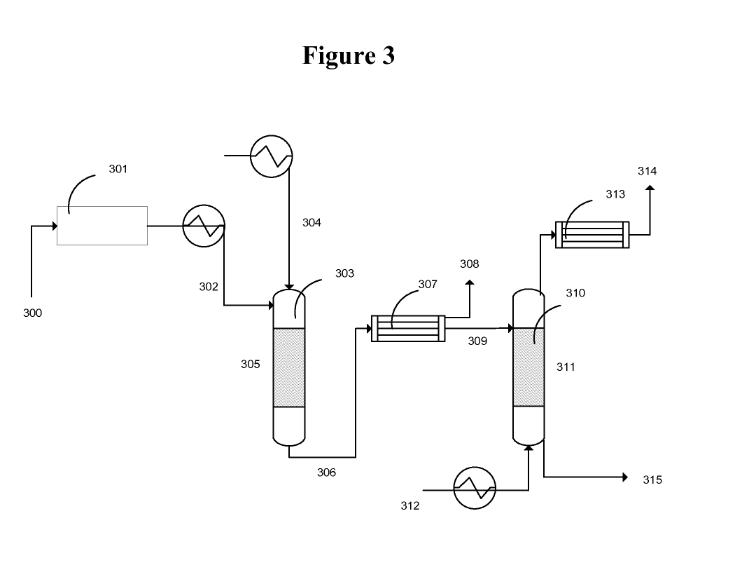

FIG. 3 provides different configuration of dehydration vessel and the esterification vessel in the manufacture of acrylic acid ester using lactic acid as the source material. In this configuration, highly purified lactic acid (302) derived from fermentation broth containing ammonium lactate (300) through a conversion process (301) is first fed into a dehydration vessel (303) from the top. The dehydration vessel contains solid dehydration catalyst on a solid support (305). An inert gas stream (304) is also introduced in to the dehydration vessel from the top. The inert gas stream purges the acrylic acid resulting from the dehydration of lactic acid (306) into a condenser (307) along with the water released from the dehydration reaction. Through differential distillation, acrylic acid is recovered (309) from total condensate from the dehydration vessel while the water is released as water vapor (308). The recovered acrylic acid (309) is introduced into the esterification vessel (310) as a source material from the top. The esterification vessel contains solid esterification catalyst on a solid support (311). Alcohol vapor (312) introduced into the esterification vessel initiates the esterification of acrylic acid on the surface of the catalyst leading to the formation of acrylic acid ester (315) which is collected at the bottom of the esterification vessel and appropriately processed to recover acrylic acid ester for further purification. The excess amount of alcohol and water vapor is released from the top of the esterification vessel and properly collected and recovered through condensation (313) for further recycling (314) into the esterification vessel.

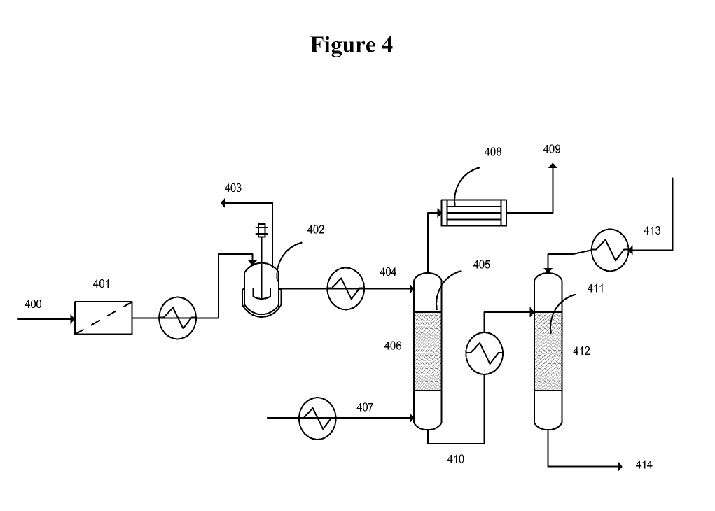

Shown in FIG. 4 are steps for recovering lactide from a fermentation broth containing ammonium lactate (400) and subsequent conversion to acrylic acid ester. The fermentation broth is filtered (401) to remove particulate matter and pumped into the heated vessel (402) to evaporate water. During this concentration process, ammonium lactate is also split leading to the release of ammonia gas (403). The ammonia gas thus released is captured and recycled to the fermentation vessel as ammonium hydroxide in order to maintain the neutral pH during fermentative production of lactic acid. With the release of ammonium, the free lactic acid formed undergoes condensation reaction to form lactide (404). The lactide thus formed (404) is introduced into an esterification vessel (405) on the top as a source material. Esterification vessel contains solid esterification catalyst on a solid support (406) and maintained at elevated temperature under atmospheric pressure. Alcohol vapor (407) is introduced into the esterification vessel from the bottom. The excess amount of alcohol vapor escaping from the esterification vessel is captured by a condenser unit (408) and recycled (409) into the esterification vessel as alcohol vapor. The lactic acid ester and the water resulting from the esterification reaction (410) are collected from the bottom of the esterification vessel and introduced into the dehydration vessel (411) as a source material at the top. The dehydration vessel contains solid dehydration catalyst on a solid support (412). A stream of inert gas (413) is purged through the dehydration vessel from the top. The acrylic acid ester and the water (414) resulting from the dehydration reaction are collected from the bottom of the dehydration vessel and acrylic acid ester is recovered through differential distillation.

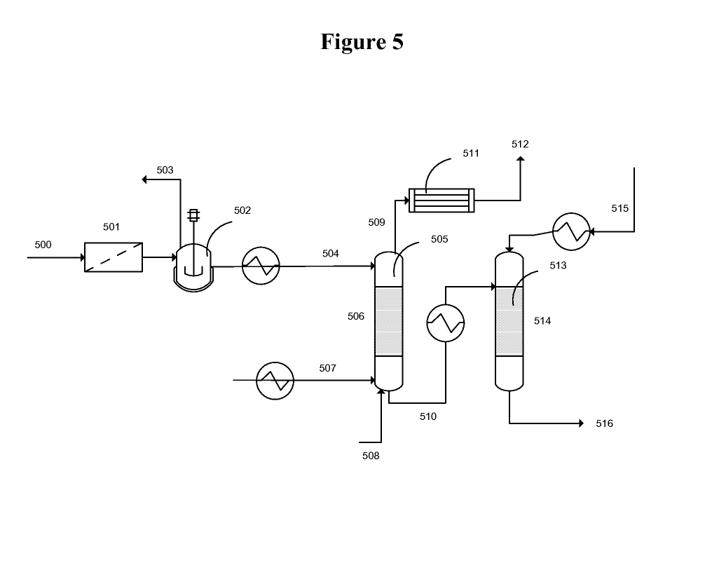

FIG. 5 illustrates another preferred equipment configuration for manufacturing acrylic acid ester from the fermentation broth containing ammonium lactate (500). The fermentation broth containing ammonium lactate is filtered through a filtration unit (501) to remove particulate matter and is pumped into a heating vessel (502) to evaporate the water and increase the ammonium lactate concentration. The ammonia released (503) during this evaporation step is captured and recycled as ammonium hydroxide to the fermentation vessel in order to maintain the neutral pH during fermentative production of lactic acid. The concentrated ammonium lactate solution (504) is fed into an esterification vessel (505) as a source material. The esterification vessel does not have any esterification catalyst and contains only a solid support (506) for the esterification reaction to occur. Alcohol vapor (507) is supplied to the esterification vessel from the bottom. A stream of inert gas (508) is also purged into the esterification vessel from the bottom. The esterification vessel is maintained at an elevated temperature and at that elevated temperature, there is a release of ammonia along with water and alcohol vapor from the top of the esterification vessel (509). The ammonia gas thus released is captured as ammonium hydroxide (511) solution which is recycled (512) back to the fermentation vessel. The alcohol vapor can also be recaptured and recycled into the esterification vessel. Lactic ester and the water released from the esterification reaction (510) are collected from the bottom of the esterification vessel and fed into the dehydration vessel (513) at the top. The dehydration vessel contains dehydration catalyst on a solid support (514). An inert gas stream (515) is purged into the dehydration vessel form the top. Acrylic acid ester, water, inert gas and other reaction products (516) are collected at the bottom of the dehydration vessel and the acrylic acid ester is recovered through differential distillation.

The present invention may be more fully understood from the following examples which are offered by way of illustration and not by way of limitation.

### Example 1

### Preparation of Fermentation Broth Containing Lactic Acid

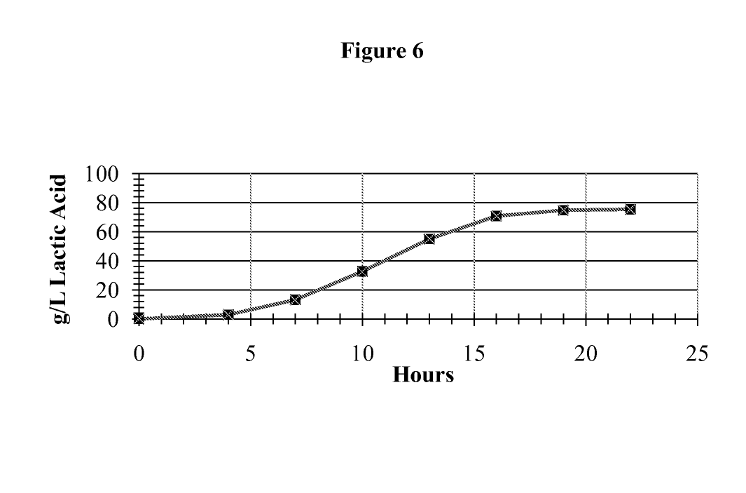

TG160 stain of E. coli was grown in a minimal mineral medium supplemented with 100 g of glucose per liter in a 20 L fermentor. Initial inoculum was grown in 1×NBS medium supplemented with 100 mM MOPS pH 7.4, 2% glucose, 1 mM MgSO₄, 1×TE, and 0.1 mM CaCl₂. The growth medium also contained 180 ml of 1M KH₂PO₄, 13 ml of 1.5 M MgSO₄, 13 ml of 1M Betaine, and 65 ml of 1000× Trace mineral stock. The 1000× trace mineral stock contained 1.6 g FeCl₃:6H₂O, 0.2 g CoCl₂:6H₂O, 0.1 g CuCl₂. 2H₂O, 0.2 g ZnCl₂, 0.2 g Na₂MoO₄:2H₂O, 0.55 g MnCl₂: 4H₂O, 0.05 g H₃BO₃, and 10 ml of HCl (Conc) in a total volume of 1000 ml. Fermentor was maintained at 37° C. and 9N NH₄OH was used to maintain the pH at 7.0 during the course of 22 hours of growth in the fermentor. The level of lactic acid, succinic acid, fumaric acid, malic acid, acetic acid and pyruvic acid were measured at different points during the 22 hour long fermentation. There was a steady increase in the lactic acid level reaching a maximum level of 75.3 g/L at 22 hours (FIG. 6). The lactic acid yield as the percentage of moles of lactic acid produced to the number of moles of glucose consumed was found to be 89.50%. The pyruvic acid and acetic acid concentrations were 0.03 g/l and 0.44 g/l respectively. The other organic acids such as malic acid, fumaric acid and succinic acid were not detectable.

### Example 2

### Gas Chromatographic Analysis

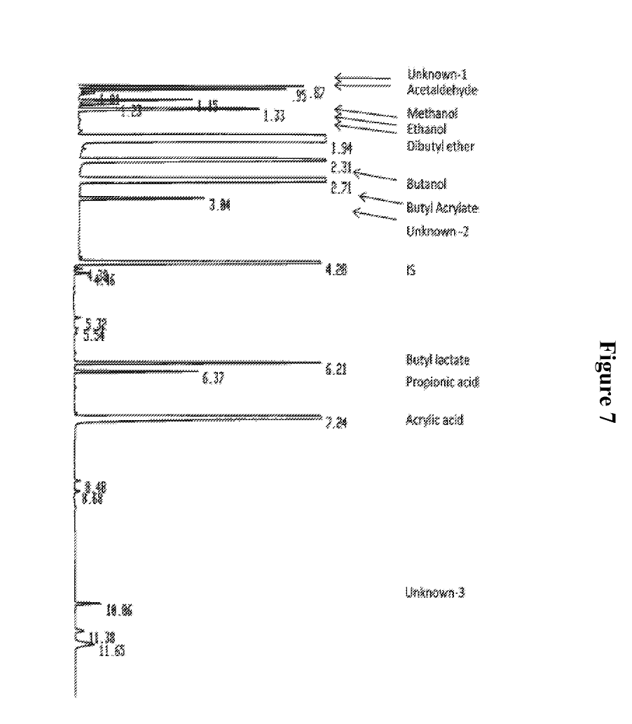

Gas chromatographic analysis was followed to quantify the various components in the starting solutions and reaction products. As a way of establishing a calibration standard, a mixture containing acetaldehyde, methanol, ethanol, dibutylether, butanol, butyl acrylate, butyl lactate and acrylic acid was analyzed using HP 5890 gas chromatographic apparatus with FID detector and an electronic integration. Capillary column HP FFAP (25 m×0.32 mm×0.50 mkm) was used. The instrument conditions were as follows. Split vent: 20 ml/min; Air flow: 300 ml/min; Hydrogen flow: 30 ml/min; Head pressure: 15 psi; Signal Range: 9; Injection volume: 0.5 mkl. The following temperature program were used. Initial temperature: 80° C., hold 2 minutes, ramp 15° C./min to 150° C., hold 1 min; Program A: ramp 20° C./min to 190° C. hold 6 min; Injection and detector temperatures: 200° C. and 220° C. Diethylene glycol dimethyl ether in dioxane (0.3079 g/g) was used as the internal standard. As shown in the FIG. 7, components in the starting solution and reaction products are clearly separated in the gas chromatographic profile.

### Example 3

### Conversion of Methyl Lactate to Methyl Acrylate

The dehydration efficiency of various catalysts was tested using methyl lactate as the starting material. Methyl lactate (>98% purity) was purchased from TCI America and used without further purification. Methanol used as reaction solvent was obtained from Mallincrodt. The 4-methoxyphenol was obtained from TCI America and was added into the liquid feed at 100 ppm as an inhibitor. The acrylic acid, the methyl acrylate, 2-methoxy propionic acid methyl ester (2-MOPAME) used as GC calibration standards were purchased from TCI America.

Six different catalysts were tested for their efficiency in dehydration reaction using methyl lactate as the substrate. Zeolite 13X-Na (Math 13X-Na) catalyst was obtained from Matheson Coleman and calcined in electrical furnace in static air at 500° C. for 12 hours. The catalyst was transferred to a desiccator and kept in screw vials under vacuum until use. Cesium acetate impregnated Math 13X-Na (Math 13X-Na—Cs⁺) was prepared by means of treating 15 g of Math 13X-Na with 21 ml of deionized water containing 1.5 g of Cesium Acetate overnight. The water was removed by rotary evaporation at 55° C. under vacuum and solid material was transformed into a crucible and calcined at 500° C. for three hours. Grace 13XNaCs11 catalyst was prepared by means of adding 15 g of Grace 13X zeolite catalyst to 30 ml solution of Cesium Acetate (1.08 g in 30 ml water) and leaving it overnight. Next day, the water was decanted and zeolite was washed once with 30 ml of water, rotary evaporated under vacuum at 60° C. and calcined at 500° C. for 3 hours. Grace 13XNaCs22 catalyst was prepared by means of adding 15 g of Grace 13X zeolite catalyst to 30 ml solution of Cesium Acetate (2.16 g in 30 ml water) and leaving it overnight. Next day, the water was decanted and zeolite was washed once with 30 ml of water, rotary evaporated under vacuum at 60° C. and calcined at 500° C. for 3 hours. 13X-NaCs Ex catalyst was prepared by adding 30 grams of Grace 13X zeolite in Sodium form with 400 ml of aqueous Cesium Acetate (4.3 g in 60 cc water) and stirring slowly in a rotary-evaporator for 18 hours. After 18 hours of stirring, the supernatant was removed and replaced with fresh Cesium acetate solution and the procedure repeated for 2 more times. The zeolite was suction filtered and washed in the filter under constant layer of water. About 1.2 L of water was used for washing. The zeolite was dried in oven at 100° C. for 18 hours and then calcimined at 500° C. for 3 hours. 13X-NaCsRu catalyst was prepared by adding fifteen grams of Grace 13X zeolite in Na form slowly to 30 mL aqueous CsAcetate (4.33 g in 30 cc water) and were soaked overnight. On the next day the water was decanted and the zeolite washed once with 30 cc water. Rot-evaporated under vacuum (60° C.) and the zeolite calcined 500 C for 3 hours. Subsequently, the sieve was soaked in ethanolic solution of RuCl₃ (0.172 g RuCl₃ into 30 ml ethanol), dried under reduced pressure without washing the catalyst and calcined also 500 C for 3 hours to deposit 0.5% Ru.

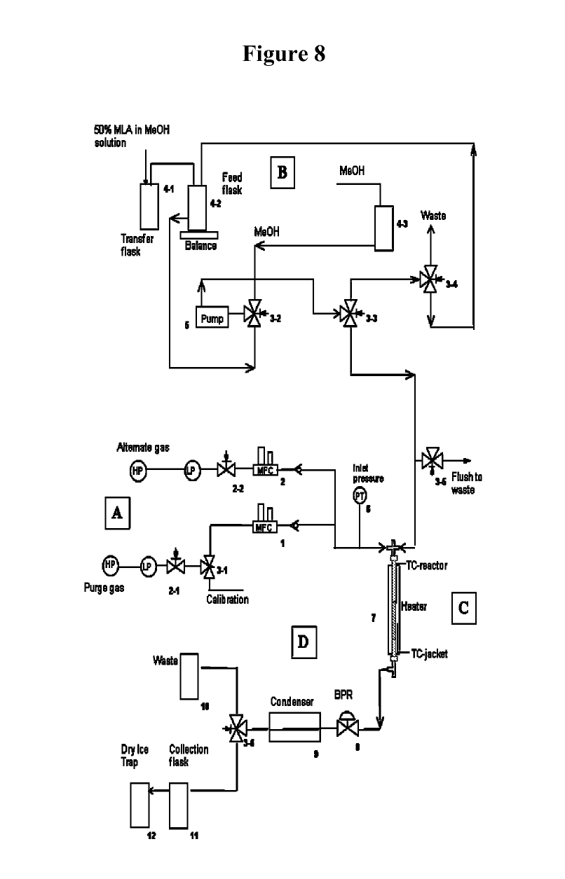

The continuous vapor phase dehydration of methyl lactate in methanol or water as a reaction solvent over solid catalysts was performed in fixed bed reactor system as shown in FIG. 8. The reactor is made of ½″×12″ stainless steel tube which was first packed with three 10 mkm stainless steel Inlet solvent filters (see for example Cat #A-302, Upchurch Scientific), serving as support for the catalyst bed. The middle section of the reactor was packed next with 10.5 mL of particulate catalyst using a GC column packing vibrator. The top section of the reactor accommodates four of the same inlet filters, providing 8 cc porous stainless steel contact space which was used as pre-evaporation and gas-liquid mixing section. The reactor tube was placed in a Flatron CH 30 column heater, which was retrofitted with high power heating tape (Omega, 470W, Part #STH051-060). The temperature in the column heater was monitored by a TC inserted near the internal wall of the heater and controlled by a temperature controller (model M 260, J-KEM Scientific). The reactor pressure was also monitored by a gage at the reactor inlet port. The liquid hourly space velocity (LHSV) was varied in relatively narrow range of 0.50-1.10 h⁻¹, (based on 10.5 mL catalyst volume and 0.1-0.2 cc/min liquid flow rate) while the nitrogen flow rate was varied in the 4.4-5.6 cc/min range. The feed solution used in all of the test runs was 50% wt Methyl lactate in either methanol or water. The combined gas-liquid flows after the reactor were sent to a gas liquid separator to remove the nitrogen from the liquid phase. Combined sample was collected at specified time points and analyzed using GC apparatus. The results are shown in Table 1.

Table 2 shows the results of the experiments done to determine the effect of water as an additive to the solvent composition. Catalyst UOP 13X-Na ( 1/16″ extrudate) was crushed and sieved through 40-60 mesh particle size. It was calcined in oven at 450° C. by slow temperature over 2 hours and held at 450° C. for 2 hours. No pre-activation of the catalyst in the reactor was performed. 5 cc of the catalyst was charged in the fixed bed reactor and heated to the reaction temperature over 1 hour in nitrogen flow of 5 cc/min. LHSV of methyl lactate solution was 1.2 h⁻¹. The reaction temperature was 300° C. and the gas flow rate was 5 cc/minute. As the results shown in Table 2 indicate, with the increase in the water content, the selectivity for acrylic acid increased.

Table 3 shows the results from the experiment conducted to determine the effect of methyl lactate concentration in water as the reaction solvent. Catalyst UOP 13X-Na ( 1/16″ extrudate) was crushed and sieved through 40-60 mesh particle size. It was calcined in oven at 450° C. by slow temperature over 2 hours and held at 450° C. for 2 hours. 5 cc of the catalyst was charged in the fixed bed reactor and heated to the reaction temperature over 1 hour in nitrogen flow of 5 cc/min. Feed rate of methyl lactate was 3.185 g/h.

### Example 4

### Sequential Esterification and Dehydration Reaction

In this example lactic acid was used as the source material. In the first stage lactic acid was esterified to methyl lactate using 4.2 mL of Amberlyst 70 (wet) resin contained in a fixed bed reactor set up described in Example 3. The liquid feed was prepared by dissolving 272 g of 85% pure lactic acid in 164.3 g of methanol and 43.63 mg of 4-methoxyphenol. The reaction conditions were as follow; temperature=140° C., feed flow=0.3 cc/min, contact time=5.6 s, methanol to lactic acid ratio=2. The results are shown in Table 4.

In the next stage, the liquid feed composition from first stage was subjected to dehydration reaction in the presence of four different dehydration catalysts. The liquid feed composition recovered from the 1^{st} stage esterification reaction is: 18.3 wt % of methanol, 41.3 wt % of Methyl Lactate, 16.7 wt % of water and 5.7 wt % of Lactic acid. No pre-activation of the catalyst was used for these catalysts. The catalysts were heated to the reaction temperature over 1 hour with 5 cc/min of argon flow. The reaction conditions were: feed flow=0.1 cc/min, LHSV=1.2 h⁻¹, argon flow=5 cc/min, temperature=300° C. Four different catalysts were prepared as described below.

Grace 13X-Na: Thirty grams of Grace 13X zeolite in sodium form were added slowly to 800 mL of 1.5M NaCl solution and the suspension stirred at 30° C. for overnight. The resulting sample was then decanted and washed multiple times until free of Cl⁻, dried initially at 30° C. at 2-3 mm vacuum for 2 hours and at 60° C. (also at 2-3 mm Hg) for 6 h. The zeolite was transferred into vacuum oven at 120° C. and dried under 15 mm Hg for overnight. Calcined for 4 h at 450° C. by slowly ramping the temperature to 450° C. for over 30 min. The Grace 13X-Na was calcined at 500° C. for 3 hours before use.

UOP 13X-Na (as is): The 1/16″ extrudates were crushed and sieved trough 40-60 mesh particle size. It was calcined in oven at 450° C. by slow temperature ramp over 2 hours, held at 450° C. for 2 hours.

Grace 13X-K: Thirty grams of Grace 13X zeolite in Na form were added slowly to 200 mL of KCl solution (5.53 g of KCl in 200 mL of water) and the suspension stirred at 30° C. for overnight. The resulting sample was then decanted and washed multiple times until free of Cl⁻, dried initially at 30° C. at 2-3 mm vacuum for 2 hours and at 60° C. (also at 2-3 mm Hg) for 6 h. The zeolite was finally calcined for 3 h at 500° C. by slowly ramping the temperature to 500° C. for over 30 min.

Tosoh Na-L zeolite: 15 grams of Tosoh K-L zeolite were added slowly to 200 mL of 0.6 M NaCl (7.01 g in 200 ml water) solution and the suspension stirred at 30° C. for overnight. The resulting sample was then decanted and washed multiple times until free of Cl⁻, dried initially at 30° C. at 2-3 mm vacuum for 2 hours and at 60° C. (also at 2-3 mm Hg) for 4 h. The zeolite was calcined at 450° C. for 3 h by slowly ramping the temperature to 450° C. for over 30 min.

The results from the sequential esterification and dehydration reactions are shown in Table 5.

### Example 5

### Esterification of Lactide to Butyl Lactate

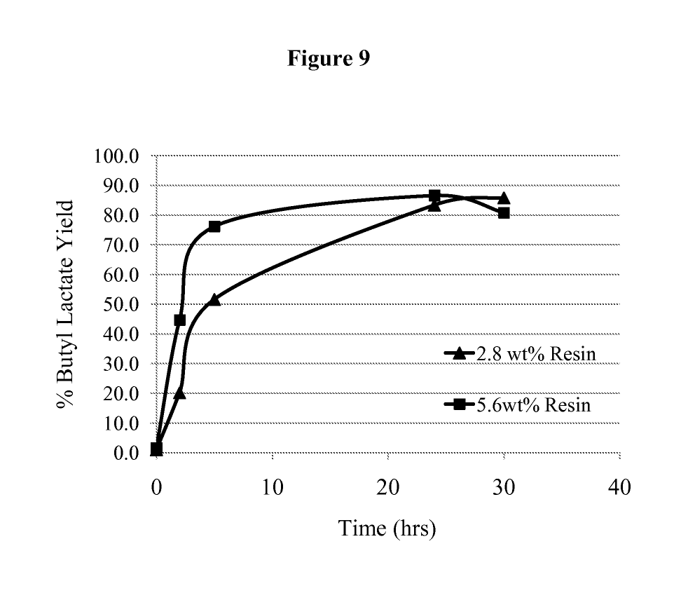

In this example we used lactide as the source material in the esterification reaction. Lactide was purchased from Sigma Aldrich and n-butanol was from Fisher Scientific. Two reaction vessels were set up using 100 mL media bottles with screw cap and the reaction heat was provided by a glycerol bath on a hot stir plate. 10 grams of lactide was added to 25.7 g of butanol. The reaction was run with Amberlyst-36 as catalyst in two different concentrations (1 gram of Amberlyst-36 to one vessel and 2 g of Amberlyst-36 to another vessel). Agitation was provided by a stir bar at 380 rpm. Frequent time point samples were collected to monitor the reaction progress and the samples were analyzed by HPLC & GC. The results are shown in Table 6 and in FIG. 9.

### Example 6

### Esterification of D,L-Lactic Acid with 1-Butanol

In this example we show the esterification of lactic acid to butyl lactate. The strongly acidic ion exchange resins A35, A45 and A70 used as catalysts in this experiment were from Rohm and Haas. The resins were used either in wet form or were dried under vacuum in Roto-vaporator for 2 h at 50° C. and the volume and the density of the dry material was calculated. The D,L-Lactic acid was purchased from Aldrich and TCI. The D,L-Lactic acid obtained from supplier was reported to be 85% pure. Titration of the material received from the supplier with 0.1N NaOH was found to be 72% pure suggesting that the rest of the lactic acid in the commercial supply exist in oligomeric forms. Under the esterification conditions used in the present invention, the oligomeric forms will also undergo esterification yielding butyl lactate. The experiments were run in the fixed bed tubular reactor system described in Example 3.

The feed solution in this cycle of experiments was made by dissolving 100 g of 72% Lactic acid (0.8 M determined by titration with NaOH) into 1-Butanol (140 g, 1.89M). The feed solution was additionally spiked with 0.025 g (100 ppm) of 4-Methoxy phenol used as polymerization inhibitor. The transfer flask was purged 3 times by alternately pressurize-release with 8 psi nitrogen to remove the dissolved oxygen. The nitrogen mass flow controller (MFC-1) was set to 4.4 cc/min and the heating of the reactor initiated. When the temperature reached 70° C., the liquid flow was started at the pre-set flow rate. The volume and the weight of each collection were recorded and aliquot titrated with 0.1N NaOH to determine the remaining Lactic acid. A second sample was also taken and analyzed by GC. Results are shown in Table 7.

### Example 7

### Esterification of Ammonium Lactate to Butyl Lactate

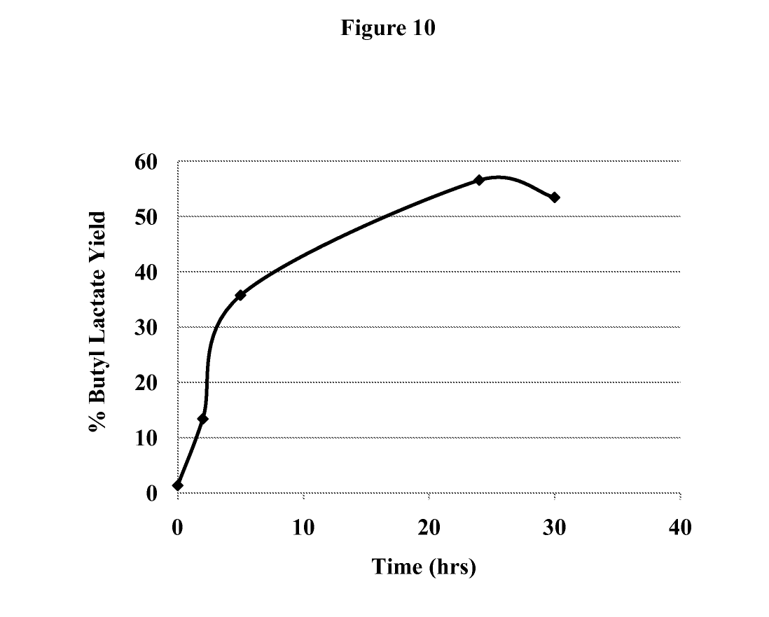

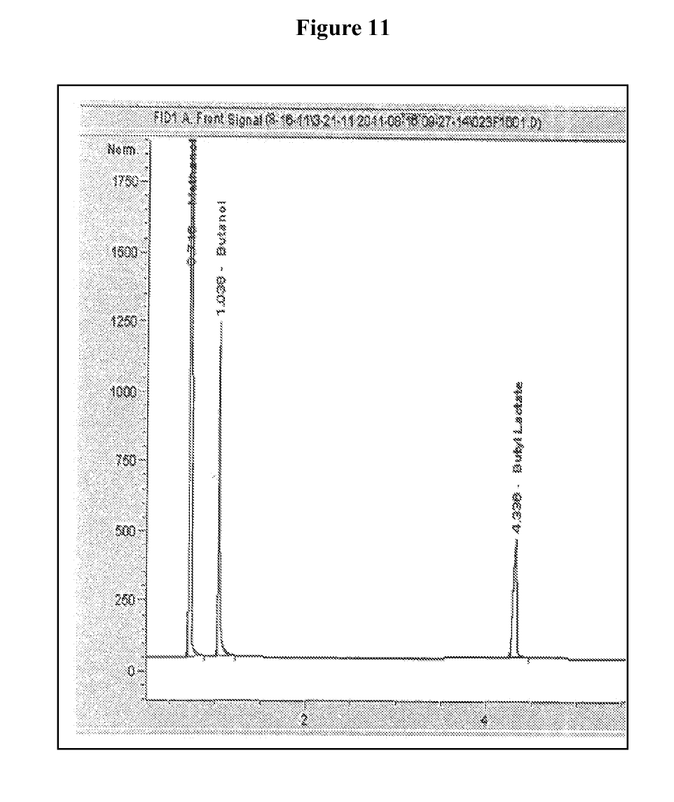

In this experiment we show the formation of butyl lactate when ammonium lactate is treated with n-butanol. Same reaction set up described in Example 5 was used to perform this reaction. 9 g of concentrated ammonium lactate was used as feed, which was prepared by evaporating excess water from 125 g of fermentation broth containing 61.2 g of equivalent lactic acid. 18.2 g of n-butanol was added to 9 g of concentrated ammonium lactate solution and esterification was carried out in the absence of catalyst at 105° C. Sample aliquots were collected using a syringe needle and analyzed by GC & HPLC. The results are shown in FIGS. 10 and 11 and in Table 8.

### Example 8

### Dehydration of Butyl Lactate to Butyl Acrylate and Acrylic Acid

In this experiment we show the formation of butyl acrylate and acrylic acid from the dehydration of butyl lactate. The composition of liquid feed was: 50% Butyl Lactate in 45% Butanol and 5% water (200 g Butyl Lactate, 180 g Butanol, 20 g water, 40 mg 4-methoxyphenol). The reaction conditions were: feed flow rate: 0.1 cc/min, contact time (CT 4.8 s; volume of catalysts—5 cc. Three different catalysts namely Tosoh K-L zeolite as is, Tosoh sodium exchanged K-L zeolite, and Tosoh Cesium exchanged K-L zeolite were tested. The preparations of these catalysts are as follows:

Tosoh K-L zeolite (as is): The 1/16″ extrudates were crushed and sieved trough 30-60 mesh particle size. It was calcined in oven at 450° C. by slow temperature ramp over 2 hours, hold at 450° C. for 2 hours.

Tosoh sodium exchanged K-L zeolite: 15 grams of Tosoh K-L zeolite were added slowly to 200 mL of 0.6 M NaCl (7.01 g in 200 ml water) solution and the suspension stirred at 30° C. for overnight. The resulting sample was then decanted and washed multiple times until free of Cl⁻, dried initially at 30° C. at 2-3 mm vacuum for 2 hours and at 60° C. (also at 2-3 mm Hg) for 4 h. The zeolite was calcined at 450° C. for 3 h by slowly ramping the temperature to 450° C. for over 30 min.

Tosoh Cesium exchanged K-L zeolite: 15 grams of K-L zeolite were added slowly to 200 mL of 0.6 M aqueous Cs-Acetate solution (23.03 g in 200 cc water) and the RB flask stirred slowly at RT for 18 hours. The supernatant was removed and replaced and washed with water 3-4 times. The zeolite was dried initially at 30° C. at 2-3 mm vacuum for 2 hours and at 60° C. (also at 2-3 mm Hg) for 4 h. The zeolite was calcined at 450 C for 3 h by slowly ramping the temperature to 450 C for over 30 min.

The experiments were run in the fixed bed tubular reactor system described in Example 3. The results are shown in Table 9.

### REFERENCES

All references are listed for the convenience of the reader. Each reference is incorporated by reference in its entirety.
U.S. Pat. No. 2,183,357
U.S. Pat. No. 2,464,364
U.S. Pat. No. 2,859,240
U.S. Pat. No. 3,022,336
U.S. Pat. No. 3,087,962
U.S. Pat. No. 4,631,264
U.S. Pat. No. 4,729,978
U.S. Pat. No. 4,786,756
U.S. Pat. No. 5,068,399
U.S. Pat. No. 5,071,754
U.S. Pat. No. 5,250,729
U.S. Pat. No. 5,252,473
U.S. Pat. No. 5,268,506
U.S. Pat. No. 5,371,273
U.S. Pat. No. 6,545,175
U.S. Pat. No. 6,992,209
U.S. Pat. No. 7,538,247
U.S. Pat. No. 7,629,162
U.S. Pat. No. 7,687,661
U.S. Pat. No. 7,696,375
U.S. Pat. No. 7,851,188
U.S. Pat. No. 7,910,342
U.S. Pat. No. 7,993,889
U.S. Patent Application Publication No. US2010195505
U.S. Patent Application Publication No. US20100129886
U.S. Patent Application Publication No. US20100221801
U.S. Patent Application Publication No. US20100062505
U.S. Patent Application Publication No. US20110183392
U.S. Patent Application Publication No. US20110159558
U.S. Patent Application Publication No. US20110195505
U.S. Patent Application Publication No. US 20110112334
European Patent Application Publication No. 0,614,983 A2
Patent Abstracts of Japan Publication No. 2003-284580
Patent Abstracts of Japan Publication No. 2004-315411
International Patent Application Publication No. WO/2011/040700
Aida, T. K., Ikarashi, A., Saito, Y., Watanabe, M., Smith Jr., R. L., and Arai, K. (2009) J. Supercritical Fluids 50: 257-264.
Alvarez, M. E. T., Moraes, E. B., Machado, A. B., Filho, R. M., Wolf-Maciel, M. R. (2007) Evaluation of liquid-liquid extraction process for separating acrylic acid produced from renewable sugars. App. Biochem. Biotech. 136-140: 451-462.
Danner, H., Urmos, M., Gartner, M., and Braun, R. (1998) Biotechnological production of acrylic acid from biomass. App. Biochem. Biotechnol. 70-72: 887-894.
Fan, Y., Zhou, C., and Zhu, X. (2009) Selective catalysis of lactic acid to produce commodity chemicals. Catalysis Review 51(3): 293-324)
Glauser, J., Blagoev, M., and Kumamoto, T. (2011) CEH Marketing Research Report. Acrylic acid, acrylate esters and superabsorbent polymers.
Gunter, G. C., Langford, R. H., Jackson, J. E., Miller, D. J. (1995) Catalysts and supports for conversion of alctic acid to acrylic acid and 2,3-pentanedione. Ind. Eng. Chem. Res. 34: 974-980.
Iizuka, T., Fujie, S., Ushikubo, T., Chen, Z-h., Tanabe, K. (1986) Esterification of acrylic acid with methanol over niobic acid catalyst. App. Catal. 28: 1-5.
Jiang, X., Meng, X. and Xian, M. (2009) Biosynthetic pathway for 3-hydroxy propionic acid production. Appl. Microbiol. Biotechnol. 82: 995-10030.
Jinfeng, Z., Jianping, L., Xiaobo, X. and Peilin, C. (2008) Evaluation of catalysts and optimization of reaction conditions for the dehydration of methyl lactate to acrylates. Chin. J. Chem. Eng. 16: 263-269.
Lira, C. T and McCrackin, P. J. (1993) Ind. Eng. Chem. Res. 32: 2608-2613
Lunelli, B. H., Rivera, E. C., Vasco de Toledo, E. C., Macial, M. R. W., and Filho, R. M. (2008) Appl. Biochem. Biotechnol. 148: 175-187
Mok, W. S-L., and Antal Jr. M. J. (1989) Formation of acrylic acid from lactic acid in supercritical water. J. Org. Chem. 54: 4596-4602.
Shi, H. F., Hu, Y. C., Wang, Y., Huang, H. (2007) KNaY-zeolite catalyzed dehydration of methyl lactate. Chin. Chem. Lett. 18: 476-478.
Straathof, A. J. J., Sie, S., Franco, T. T. (2005) Feasibility of acrylic acid production by fermentation. Appl. Microbiol. Biotechnol. 67: 727-734.
Sun, P., Yu, D., Fu, K., Gu, M., Wang, Y., Huang, H. and Ying, H. (2009) Potassium modified NaY: A selective and durable catalyst for dehydration of lactic acid to acrylic acid. Catal. Comm. 10: 1345-1349.
Wadley, D.C., Tam. M. S. Kokitkar, P. B., Jackson, J. E., Miller, D. J. (1997) Lactic acid conversion to 2,3-pentanedione and acrylic acid over silica-supported sodium nitrate: Reaction optimization and identification of sodium lactate as the active catalyst. J. Catal. 165: 162-171.
Wang, H., Yu, D., Sun, P., Yan, J. Wang, Y. and Huang, H. (2008) Rare earth metal modified NaY: Structure and catalytic performance for lactic acid dehydration to acrylic acid. Catal. Comm. 9: 1799-1803.
Yang, Jung-II, Cho, S—H., Kim, H-J., Joo, H., Jung, H., Lee, K-Y. (2007) Production of 4-hydroxybutyl acrylate and its reaction kinetics over amberlyst 15 catalyst. Can. J. Chem. Eng. 85: 83-91.
Zhang, J., Lin, J., Cen, P. (2008) Catalytic dehydration of lactic acid to acrylic acid over sulfate catalysts. Can. J. Chem. Eng. 86:1047-1053.
Zahng, Z., Qu, Y. Wang, S, and Wang, J. (2009) Catalytic performance and characterization of silica supported sodium phosphates for the dehydration of methyl lactate to methyl acrylate and acrylic acid. Ind. Eng. Chem. Res. 48: 9083-9089.
Xhang, Z., Qu, Y., Wang, S., Wang, J. (2010) Theoretical study on the mechanisms of the conversion of methyl lactate over sodium polyphosphate catalysts. J. Mol. Catal. A: Chem. 323: 91-100.
Xu, X., Lin, J. and Cen, P. (2006) Advances in the research and development of acrylic acid production from biomass. Chinese J. Chem. Eng. 14: 419-427.
Zhang, J., Zhao, Y., Pan, M., Feng, X., Ji, W., Au, C-T. (2011) Efficient acrylic acid production through bio lactic acid dehydration over NaY Zeolite modified by alkali phosphates. ACS Catal. 1: 32-41.

TABLE 1
Conversion of methyl lactate to methyl acrylate

| | | Gas Flow | | Selectivity (mole %) | | | % |
|---|---|---|---|---|---|---|---|
| | T | Rate | Conversion | Methyl | Acrylic | | Mass |
| Catalyst | (° C.) | (cc/min) | (%) | Acrylate | Acid | 2MOPAME | Loss |
| Math 13X—Na | 320 | 4.4 | 22 | 23.9 | 8.5 | 40.3 | 4.4 |
| (500° C./12 hrs) | | | | | | | |
| Math 13X—Na—Cs+ | 320 | 4.4 | 59.4 | 17.6 | 8.5 | 11.4 | 7.9 |
| (500° C./3 hrs) | | | | | | | |
| Grace13XNaCs11 | 290 | 4.4 | 53.3 | 30.3 | 9.7 | 12.3 | 4 |
| (500° C./3 hrs) | | | | | | | |
| Grace13XNaCs22 | 300 | 4.4 | 70.6 | 45.1 | 5 | 10.7 | 3.7 |
| (500° C./3 hrs) | | | | | | | |
| 13X—NaCs-Exch | 300 | 0.1 | 3.2 | 33.6 | 18.2 | 64.3 | 5.7 |
| (500° C./3 hrs) | | | | | | | |
| 13X—NaCsRu | 300 | 5 | 34.2 | 37.8 | 13.5 | 15.9 | 6.2 |
| (500° C./3 hrs) | | | | | | | |

TABLE 2
Effect of water as an additive to the solvent composition

| | | Methyl | Liquid | | Molar Selectivity (%) | | | % |
|---|---|---|---|---|---|---|---|---|
| Water | Methanol | Lactate | Flow Rate | Conversion | Methyl | Acrylic | | Mass |
| (wt %) | (wt %) | (wt %) | (cc/min) | (%) | Acrylate | Acid | 2MOPAME | Loss |
| 4.8 | 47.6 | 47.6 | 0.1 | 47.1 | 27.6 | 14.8 | 19 | 6.2 |
| 33.3 | 19 | 47.6 | 0.1 | 45.9 | 8.5 | 43.2 | 5.1 | 10.9 |
| 42.9 | 9.5 | 47.6 | 0.1 | 54.7 | 6.8 | 39.2 | 3.3 | 27.4 |
| 52.4 | 0 | 47.6 | 0.1 | 49.2 | 3.8 | 50.6 | 2 | 9 |

TABLE 3
Effect of concentration of methyl lactate in water as reaction solvent

| Water/Methyl | Liquid | | Selectivity, mol % | | | | | % |
|---|---|---|---|---|---|---|---|---|
| Lactate | Flow Rate | Conversion | | Methyl | Acrylic | | Lactic | Mass |
| (wt %) | (cc/min) | (%) | Acetaldehyde | Acrylate | Acid | 2MOPAME | Acid | Loss |
| 75/25 | 0.2 | 73.9 | 4.2 | 0 | 16.5 | 0 | 27.9 | 4 |
| 60/40 | 0.125 | 71.9 | 2.3 | 1.2 | 15 | 1 | 22.9 | 7.3 |
| 52.4/47.6 | 0.1 | 82 | 0.1 | 0.6 | 22 | 1.5 | 29 | 9 |
| 40/60 | 0.083 | 61 | 2.9 | 2.9 | 22.5 | 1.8 | 0 | 16 |
| 25/75 | 0.066 | 69.9 | 0.8 | 1.2 | 10.6 | 1.1 | 19.3 | 10.1 |

TABLE 4
Esterification of D,L-lactic acid with methanol over Amberlyst 70 (wet)

| | | Yield | | | | |
|---|---|---|---|---|---|---|
| Expt. | | Methyl | | Methyl | | |
| No | Conversion | Acrylate | 2MOPAME | Lactate | Others | Selectivity |
| 1 | 87.4 | 0.0 | 0.0 | 69.2 | 5.1 | 64.1 |
| 2 | 86.2 | 0.0 | 0.0 | 65.7 | 5.2 | 67.2 |

TABLE 5
Sequential esterification and dehydration reaction

| | | Selectivity, mole % | | | | | | % |
|---|---|---|---|---|---|---|---|---|
| | Conversion | Acrylic | Methyl | | | Lactic | | Mass |
| Catalyst ID | (%) | Acid | Acrylate | 2MOPAME | Acetaldehyde | Acid | Unknown | Loss |
| Grace 13X—Na | 31.61 | 22.4 | 0 | 0 | 0 | 2.3 | 10.7 | 9.38 |
| UOP 13X—Na | 30.05 | 46.8 | 3.8 | 2.8 | 9.9 | 4.9 | 16.1 | 1.59 |
| Grace K- | 27.4 | 9.4 | 0 | 0 | 7 | 0.6 | 1.9 | 4.1 |
| exchanged | | | | | | | | |
| 13X—Na | | | | | | | | |
| Na-exchanged | 42.92 | 57.2 | 4.8 | 4.1 | 16.4 | 6.3 | 2.3 | 4.84 |
| Tosoh | | | | | | | | |
| zeolite L-K | | | | | | | | |

TABLE 6
Esterification of lactide to butyl lactate

| mg/g | | % |
|---|---|---|
| GC | HPLC | Butyl |

| | | | | | |
|---|---|---|---|---|---|
| Time | | Butyl | Lactic | | Lactate |
| (hours) | Butanol | Lactate | Acid | Lactide | Yield |

Set-1 (2.8 wt % Resin)

| | | | | | |
|---|---|---|---|---|---|
| 0 | 725.29 | 5.61 | | 486.16 | 0.97 |
| 2 | 535.29 | 118.17 | 6.61 | 27.34 | 20.17 |
| 5 | 498.79 | 309 | 13.56 | — | 51.6 |
| 24 | 406.65 | 531.88 | 18.78 | — | 83.32 |
| 30 | 384.3 | 565.87 | 18.84 | — | 85.77 |

Set-2 (5.6 wt % Resin)

| | | | | | |
|---|---|---|---|---|---|
| 0 | 553.86 | 8.73 | | 453.74 | 1.52 |
| 2 | 431.31 | 263.32 | 19.17 | 46.48 | 44.64 |
| 5 | 387.49 | 464.64 | 27.24 | — | 76.14 |
| 24 | 295.01 | 617.92 | 21.65 | — | 86.57 |
| 30 | 219.91 | 619.00 | 21.48 | — | 80.77 |

TABLE 7
Esterification of D,L-lactic acid with 1-butanol

| Flow | Contact | | | Selectivity (%) | | | |
|---|---|---|---|---|---|---|---|
| Rate | Time | | Conversion | Butyl | Butyl | | Mass |
| (cc/min) | (s) | LHSV | % | Lactate | Acrylate | Others | Loss % |

Amberlyst 35 (wet)

| | | | | | | | |
|---|---|---|---|---|---|---|---|
| 0.518 | 6.2 | 3.88 | 65.1 | 89.9 | 0 | 10.8 | 2.03 |
| 0.296 | 10.8 | 2.22 | 67.8 | 94.4 | 0 | 12.8 | 1.78 |
| 0.1 | 32 | 0.75 | 68.9 | 99.1 | 0 | 11.3 | 0.88 |

Amberlyst 35 (dry)

| | | | | | | | |
|---|---|---|---|---|---|---|---|
| 0.495 | 6.5 | 3.71 | 63.2 | 92 | 0 | 11.9 | 0.71 |
| 0.302 | 10.6 | 2.26 | 66.1 | 94.5 | 0 | 14.5 | 1.16 |
| 0.1 | 32 | 0.75 | 69.7 | 101.9 | 0 | 12.7 | 3.51 |

Amberlyst 45 (dry)

| | | | | | | | |
|---|---|---|---|---|---|---|---|
| 0.304 | 13.6 | 2.43 | 64.8 | 83.2 | 0 | 14.8 | 0 |

Amberlyst 70 (wet)

| | | | | | | | |
|---|---|---|---|---|---|---|---|
| 0.305 | 5.5 | 4.36 | 64.1 | 87.1 | 0 | 14.3 | 0 |

TABLE 8
Esterification of ammonium lactate to butyl lactate

| mg/g | | % |
|---|---|---|
| GC | HPLC | Butyl |

| Time | | Butyl | Lactic | | Lactate |
|---|---|---|---|---|---|
| (hours) | Butanol | Lactate | Acid | Lactide | Yield |
| 0 | 592.72 | 6.04 | 147.37 | 39.59 | 1.32 |
| 2 | 551.29 | 64.01 | 154.71 | — | 13.38 |
| 5 | 502.46 | 181.93 | 112 | — | 35.72 |
| 24 | 393.47 | 408.63 | 33.85 | — | 56.54 |
| 30 | 322.58 | 455.69 | 27.02 | — | 53.43 |

TABLE 9
Dehydration of butyl lactate to butyl acrylate and acrylic acid

| | | | Selectivity (mole %) | | | | | | | Mass |
|---|---|---|---|---|---|---|---|---|---|---|
| | Temperature | Conversion | Acrylic | Butyl | Acetal- | Propionic | Dibutyl | Unkown- | Unknown- | Loss, |
| Catalyst | ° C. | % | Acid | Acrylate | dehyde | acid | ether | 1 + 2 | 3 | (%) |
| Tosoh K- | 300 | 52.6 | 23.72 | 1.85 | 5.7 | 2 | 0 | 13 | 0.9 | 12.5 |
| L, as | | | | | | | | | | |
| received | | | | | | | | | | |
| Tosoh | 300 | 50.1 | 52.4 | 3 | 10.9 | 3.99 | 0.7 | 16.8 | 1.54 | 7.55 |
| K-L, Na | | | | | | | | | | |
| exchanged | | | | | | | | | | |
| Tosoh Cs | 300 | 49.2 | 40.69 | 2.31 | 6.6 | 3.55 | 0.9 | 13.5 | 1.22 | 3.7 |
| exchanged | | | | | | | | | | |
| K-L | | | | | | | | | | |

We claim:

1. A process for producing an α,β-unsaturated carboxylic acid ester from biological feed stock, the said process comprising:
a. fermenting a biological feedstock with a biocatalyst and producing a fermentation broth comprising a hydroxy carboxylic acid or its derivatives thereof;
b. esterifying hydroxyl carboxylic acid or its derivatives in the fermentation broth with an alcohol without the addition of any exogenous esterification catalyst;
c. providing a stream of inert gas before initiating the esterification reaction with an alcohol;
d. recovering hydroxyl carboxylic acid ester; and
e. heating the hydroxyl carboxylic acid ester in the presence of a dehydration catalyst to produce an α,β-unsaturated carboxylic acid ester.

2. A process as in claim 1 wherein the hydroxyl carboxylic acid is α-hydroxy carboxylic acid or β-hydroxy carboxylic acid.

3. A process as in claim 1, wherein the derivatives of α-hydroxy carboxylic acid selected from a group consisting of ammonium lactate, sodium lactate, calcium lactate and potassium lactate.

4. A process as in claim 1, wherein the hydroxyl carboxylic acid derivative is ammonium lactate.

5. A process as in claim 1 wherein the alcohol is a C1-C10 alkly alcohol.

6. A process as in claim 1 wherein the hydroxyl carboxylic acid derivative is ammonium lactate and the alcohol is butyl alcohol.

7. A process as in claim 1, wherein the esterification reaction is carried out at above ambient temperature.

8. A process as in claim 1, wherein the dehydration catalyst is selected from a group consisting of solid acid catalyst, base catalyst, metal catalysts and molecular sieve catalysts.

9. A process as in claim 1, further comprising a step of capturing ammonia and alcohol vapor carried away by the inert gas stream and recycling ammonia in fermentation and alcohol in esterification reaction.

10. A process for preparing an ester of hydroxy propionic acid from biological stock, the said process comprising steps of:
a. fermenting a biological feedstock with a biocatalyst and producing a fermentation broth comprising a hydroxy propionic acid or its derivatives thereof;
b. heating the fermentation broth in the presence of an alcohol;
c. providing a stream of an inert gas;
d. and
e. recovering the ester of hydroxy propionic acid.

11. A process as in claim 10, wherein the hydroxy propionic acid is alpha-hydroxy propionic acid or beta-hydroxy propionic acid.

12. A process as in claim 10, wherein the derivatives of hydroxy propionic acid are selected from a group consisting of lactic acid dimer, lactic acid oligomer and inorganic salts of lactic acid.

13. A process as in claim 10, wherein the lactic acid derivative is selected from a group consisting of ammonium lactate, sodium lactate, calcium lactate and potassium lactate.

14. A process as in claim 10, wherein the lactic acid derivative is ammonium lactate.

15. A process as in claim 10, wherein the process is carried out in the presence of at least one esterification catalyst.

16. A process as in claim 10, wherein the process is carried out without the addition of any exogenous esterification catalyst.

17. A process as in claim 10, wherein the alcohol is a C1-C10 alkyl alcohol.
